(19) United States
(12) Patent Application Publication
(10) Pub. No.: US 2009/0179160 A1
(43) Pub. Date: Jul. 16, 2009

(54) SEMICONDUCTOR SUBSTRATE MANUFACTURING APPARATUS

(75) Inventors: Naoki TSUKAMOTO, Sagamihara (JP); Akihisa SHIMOMURA, Isehara (JP)

Correspondence Address: ERIC ROBINSON, PMB 955, 21010 SOUTHBANK ST., POTOMAC FALLS, VA, 20165, US

(73) Assignee: SEMICONDUCTOR ENERGY LABORATORY CO., LTD., Atsugi-shi (JP)

(21) Appl. No.: 12/344,938

(22) Filed: Dec. 29, 2008

(30) Foreign Application Priority Data
Jan. 16, 2008 (JP) 2008-007077

(51) Int. Cl. A61N 5/00

(52) U.S. Cl. 250/492.2

(57) ABSTRACT

A semiconductor substrate is manufactured with use of a semiconductor substrate manufacturing apparatus including: a cleaning portion in which a bonding surface of a base substrate, and a bonding surface of a single crystal semiconductor substrate are cleaned, wherein the single crystal semiconductor substrate includes an embrittlement region provided in a region at a predetermined depth from its surface; an electromagnetic wave irradiation portion in which the base substrate and the single crystal semiconductor substrate are attached to each other, the single crystal semiconductor substrate is irradiated with an electromagnetic wave, and the single crystal semiconductor substrate is separated using the embrittlement region as a separation plane, so that a single crystal semiconductor layer separated from the single crystal semiconductor substrate is fixed to the base substrate; and a heat treatment portion in which the single crystal semiconductor layer fixed to the base substrate is subjected to heat treatment.

### BACKGROUND OF THE INVENTION

1. Field of the Invention

The present invention relates to a manufacturing apparatus for manufacturing a semiconductor substrate having a single crystal semiconductor layer over an insulating surface. Further, the present invention relates to a method for manufacturing a semiconductor substrate having a single crystal semiconductor layer over an insulating surface with use of the manufacturing apparatus.

2. Description of the Related Art

In recent years, an integrated circuit using an SOI (silicon on insulator) substrate in which a thin single crystal semiconductor layer is formed on an insulating surface, instead of a bulk silicon wafer, has been developed. Since parasitic capacitance between a drain of a transistor and a substrate is reduced by using the SOI substrate, the SOI substrate has attracted attention as a substrate for improving performance of semiconductor integrated circuits.

As a method for manufacturing an SOI substrate, a hydrogen ion implantation separation method is known. A manufacturing method of an SOI substrate using a hydrogen ion implantation separation method is briefly described below. First, by an ion implantation method, hydrogen ions are implanted into a silicon wafer which serves as a separation substrate to form an ion-implanted layer at a predetermined depth from the surface. Then, the silicon wafer into which the hydrogen ions are implanted is bonded to another silicon wafer with a silicon oxide film interposed therebetween (bonding). After that, by heat treatment, the ion-implanted layer serves as a cleavage plane (a separation plane), and the separation silicon wafer into which the hydrogen ions have been implanted is separated as a thin film, whereby the thin film of single crystal silicon film can be formed over the another silicon wafer. The hydrogen ion implantation separation method is also called a Smart Cut (registered trademark) method.

A method in which a single crystal silicon film is formed over a base substrate made of glass by a hydrogen ion implantation separation method has been proposed (for example, Reference 1: Japanese Published Patent Application No. H11-097379).

There is proposed a method for manufacturing an SOI substrate which does not require any heat treatment at a high temperature exceeding 800° C. (e.g., Reference 2: Japanese Published Patent Application No. 2005-252244). After a semiconductor wafer is bonded to a glass substrate, heat treat is performed at a temperature not exceeding 600° C. to expand implanted ions, whereby the semiconductor wafer is separated as a thin film and a thin semiconductor layer is formed over a glass substrate. After this separation process, laser irradiation is performed to improve crystal quality of the semiconductor thin film layer and to strongly bond the semiconductor thin film layer and the glass substrate.

There is proposed a method including: dividing a silicon wafer where ions are implanted, by microwave irradiation; and cooling to 400° C. or lower to suppress damages due to a difference in thermal expansion coefficient between different materials with a temperature rise in dividing the silicon wafer (Reference 3: Japanese Published Patent Application No. 2001-244444).

Moreover, there is proposed a microwave plasma processing apparatus having a microwave generator, which includes a plurality of waveguides, in which a plurality of dielectrics are provided for the plurality of the waveguides, and by a microwave transmitted into a treatment container through the dielectrics, a certain gas is processed into plasma (Reference 4: Japanese Published Patent Application No. 2006-310794).

### SUMMARY OF THE INVENTION

Since glass substrates can have a larger area and are less expensive than silicon wafers, the glass substrates are mainly used for manufacturing liquid crystal display devices and the like. By using a glass substrate as a base substrate, a large-sized inexpensive SOI substrate can be manufactured.

However, glass substrates have disadvantages in that the glass substrates shrink when heated, have a strain point of 700° C. or lower, are more likely to sag than silicon wafers, and have undulating surfaces, and so on. Due to such disadvantages, there are more restrictions on a method for manufacturing an SOI substrate using a glass substrate as a base substrate than on a method for manufacturing an SOI substrate using a semiconductor substrate.

In a process for separating a thin film from a semiconductor substrate through the aforementioned hydrogen ion implantation separation method, the semiconductor substrate is desirably heated at a temperature of 400° C. or higher in order to expand a hydrogen gas in the semiconductor substrate. However, if the temperature of a glass substrate as well as that of the semiconductor substrate is increased in the heat treatment for separation, there is a concern that the glass substrate may shrink.

In addition, in a case where substrates are bonded to each other by a hydrogen ion implantation separation method, if a surface of a base substrate which is formed using an insulator such as a glass substrate or the like is contaminated with dusts or the like, defective bonding may occur and an area in which a single crystal semiconductor layer is not formed over the base substrate may be produced. Thus, the surfaces of the substrates to be bonded should be sufficiently cleaned in advance. If a semiconductor element such as a transistor is formed using a defective single crystal semiconductor layer having such an area in which a single crystal semiconductor layer is not formed, there is a concern that malfunction occurs. In particular, such a problem becomes noticeable in accordance with enlargement of a base substrate.

As a base substrate becomes larger, the base substrate may sag under its weight, when the base substrate is transported into or taken out of a cassette, when it is transported by a robot arm, and so on. In particular, when a base substrate to which a silicon wafer is bonded sags, the silicon wafer may be peeled off from the base substrate before heat treatment, and thus it is more difficult to transport the base substrate with the silicon wafer bonded thereto, in accordance with enlargement of the base substrate.

In view of the above problems, it is an object of the present invention to provide a semiconductor substrate manufacturing apparatus with which a single crystal semiconductor layer can be fixed to a base substrate with high accuracy even when a glass substrate, which easily shrinks, is used as the base substrate.

Moreover, it is another object of the present invention to provide a semiconductor substrate manufacturing apparatus with which occurrence of defective bonding due to surface contamination of a base substrate or a single crystal semiconductor substrate can be suppressed.

It is another object of the present invention to provide a semiconductor substrate manufacturing apparatus with which a single crystal semiconductor layer can be fixed to a base substrate with high accuracy even when a substrate which has low heat-resistance and easily sags such as a glass substrate, is used as the base substrate.

An aspect of the present invention is an appratus for manufacturing a semiconductor substrate (hereinafter, a semiconductor substrate manufacturing appratus) includes the following: a cleaning portion to conduct surface treatment of a base substrate, and a single crystal semiconductor substrate in which an insulating layer serving as a bonding layer is provided on its surface and an embrittlement region is provided in a region at a predetermined depth from its surface; an electromagnetic wave irradiation portion in which the base substrate and the single crystal semiconductor substrate are attached to each other, the single crystal semiconductor substrate is irradiated with an electromagnetic wave having a frequency of 300 MHz to 300 GHz to be heated, and the single crystal semiconductor substrate is separated using the embrittlement region as a separation plane, so that a single crystal semiconductor layer separated from the single crystal semiconductor substrate is fixed to the base substrate; and a heat treatment portion in which the single crystal semiconductor layer fixed to the base substrate is subjected to heat treatment.

An aspect of the present invention is an appratus for manufacturing a semiconductor substrate includes the following: a cleaning portion to conduct surface treatment of a base substrate, and a single crystal semiconductor substrate in which an insulating layer serving as a bonding layer is provided on its surface and an embrittlement region is provided in a region at a predetermined depth from its surface; an electromagnetic wave irradiation portion in which the base substrate and the single crystal semiconductor substrate are attached to each other, the single crystal semiconductor substrate is irradiated with an electromagnetic wave having a frequency of 300 MHz to 300 GHz to be heated, and the single crystal semiconductor substrate is separated using the embrittlement region as a separation plane, so that a single crystal semiconductor layer separated from the single crystal semiconductor substrate is fixed to the base substrate; a reprocessing portion of the single crystal semiconductor substrate, in which a surface of the single crystal semiconductor substrate from which the single crystal semiconductor layer is separated, is planarized; and a heat treatment portion in which the single crystal semiconductor layer fixed to the base substrate is subjected to heat treatment.

Moreover, the electromagnetic wave irradiation portion in each of the apparatuses for manufacturing a semiconductor substrate may include a plurality of waveguides arranged in parallel; a plurality of slots provided for each of the plurality of waveguides; and a dielectric provided to correspond to and be contact with each of the plurality of slots.

Additionally, the electromagnetic wave irradiation portion in each of the apparatuses for manufacturing a semiconductor substrate may include a plurality of waveguides; a plurality of slots provided for each of the plurality of waveguides; a dielectric provided to correspond to and be contact with each of the plurality of slots; and a stage which can move in a plane direction.

Note that the bonding layer can be formed over not only the surface of the single crystal semiconductor substrate but also the surface of the base substrate. Alternatively, the bonding layer may be formed over only a surface of the base substrate. Preferably, the bonding layer includes a barrier layer which can prevent sodium from diffusing from the base substrate side. As the barrier layer, a layer containing nitrogen, such as a silicon nitride oxide layer or a silicon nitride layer can be used.

Here, the “single crystal” means a crystal in which crystal axes extend in the same direction in any portion of a sample and which has no crystal grain boundaries between crystals. Note that, in this specification, the single crystal includes a crystal in which the direction of crystal axes are in the same direction as described above and which has no grain boundaries even when it includes a crystal defect or a dangling bond.

With use of a semiconductor substrate manufacturing apparatus according to an aspect of the present invention, a single crystal semiconductor layer can be fixed to a base substrate with high accuracy even when a substrate which easily shrinks and sags, such as a glass substrate, is used as the base substrate. Moreover, occurrence of defective bonding due to surface contamination of a base substrate or a single crystal semiconductor substrate can be suppressed.

### BRIEF DESCRIPTION OF THE DRAWINGS

In the accompanying drawings:

FIG. 1 illustrates an example of a method for manufacturing a semiconductor substrate according to Embodiment Mode 1;

FIGS. 2A and 2B illustrate an example of a semiconductor substrate manufacturing appratus according to Embodiment Mode 1;

FIGS. 3A to 3C illustrate examples of electromagnetic wave irradiation portions in the semiconductor substrate manufacturing appratus according to Embodiment Mode 1;

FIGS. 4A and 4B each illustrate an example of an electromagnetic wave irradiation portion in a semiconductor substrate manufacturing appratus according to Embodiment Mode 2;

FIGS. 5A and 5B illustrate an example of an electromagnetic wave irradiation portion in a semiconductor substrate manufacturing appratus according to Embodiment Mode 3;

FIGS. 6A to 6D illustrate an example of a method for manufacturing a semiconductor device using a semiconductor substrate;

FIGS. 7A to 7C illustrate an example of a method for manufacturing a semiconductor device using a semiconductor substrate;

FIGS. 8A to 8D are cross-sectional views illustrating an example of a method for manufacturing a thin film transistor;

FIGS. 9A to 9C are cross-sectional views illustrating an example of a method for manufacturing a thin film transistor;

FIGS. 10A to 10D are plane view illustrating an example of a method for manufacturing a thin film transistor;

FIG. 11 illustrates an example of a semiconductor device using a semiconductor substrate;

FIG. 12 illustrates an example of a semiconductor device using a semiconductor substrate;

FIGS. 13A and 13B illustrate an example of a display device using a semiconductor substrate;

FIGS. 14A and 14B illustrate an example of a display device using a semiconductor substrate;

FIGS. 15A to 15C illustrate examples of electronic devices each using a semiconductor substrate; and

FIGS. 16A to 16C illustrate an example of an electronic device using a semiconductor substrate.

### DETAILED DESCRIPTION OF THE INVENTION

### Embodiment Mode

Embodiment modes as examples of the present invention will be explained with reference to the drawings. However, the present invention is not limited to the description given below and the present invention can be carried out in many different modes, and it is easily understood by those skilled in the art that modes and details herein disclosed can be modified in various ways without departing from the spirit and the scope of the present invention. Therefore, the present invention should not be construed as being limited to the description in the following embodiment modes. Note that components denoted by the same reference numerals in different drawings are like components; therefore, repetitive descriptions on material, form, manufacturing method, and the like are omitted.

### Embodiment Mode 1

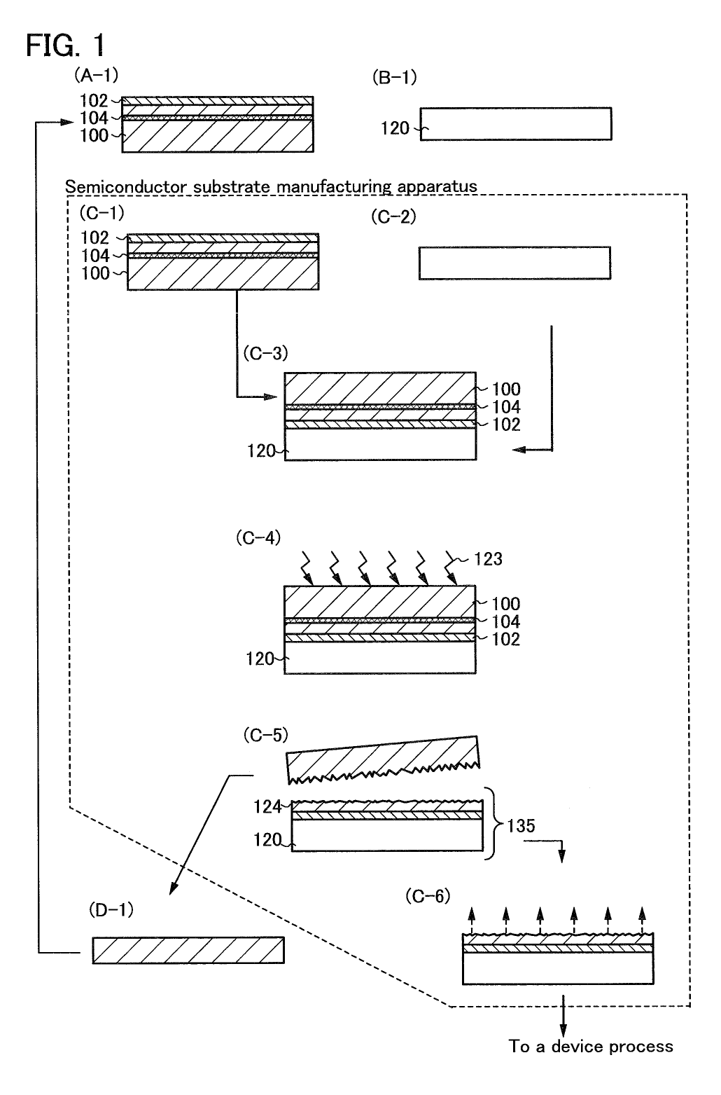

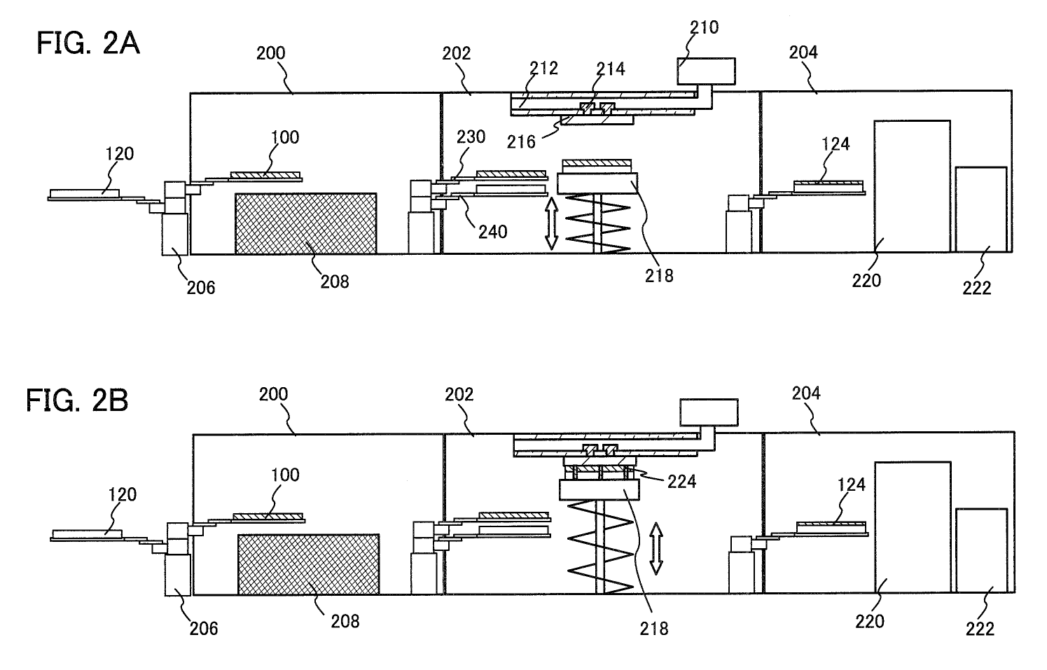

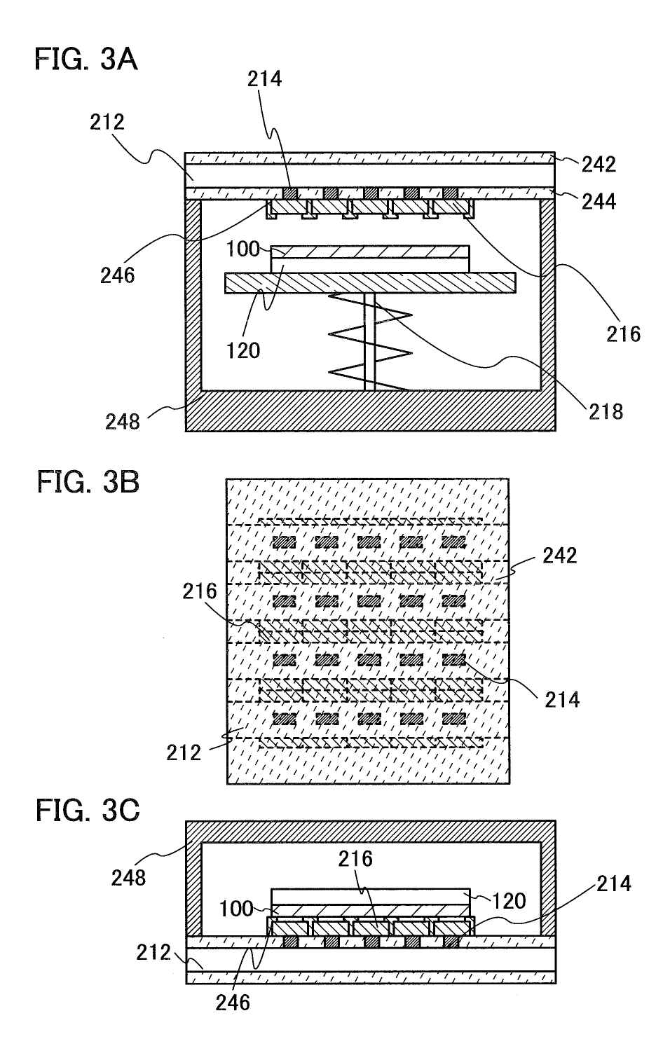

In Embodiment Mode 1, a semiconductor substrate manufacturing apparatus and a method for manufacturing a semiconductor substrate are described as an example with reference to FIG. 1, FIGS. 2A and 2B and FIGS. 3A to 3C.

FIG. 1 illustrates an example of a method for manufacturing a semiconductor substrate according to this embodiment mode.

First, a single crystal semiconductor substrate 100 is prepared (see (A-1) of FIG. 1). As the single crystal semiconductor substrate 100, a commercial semiconductor substrate can be used. For example, a single crystal silicon substrate, a single crystal germanium substrate, and a compound semiconductor substrate of gallium arsenide, indium phosphide, or the like can be used. A size of a commercial silicon substrate is typically five inches (125 mm) in diameter, six inches (150 mm) in diameter, eight inches (200 mm) in diameter, 12 inches (300 mm) in diameter, or 18 inches (450 mm) in diameter, and a typical shape thereof is a circular shape. Note that the silicon substrate is not limited to a circular shape, and a silicon substrate processed into a rectangular shape or the like can be used. In the description given below, a case in which a single crystal silicon substrate is used as the single crystal semiconductor substrate 100 is described.

After the single crystal semiconductor substrate 100 is cleaned, an insulating film is formed over a surface of the single crystal semiconductor substrate 100. Although the insulating film is not always provided, an insulating film is preferably provided in order to prevent contamination of the single crystal semiconductor substrate 100 and damages to the surface in ion irradiation to be conducted later.

Next, the single crystal semiconductor substrate 100 is irradiated with an ion beam including ions accelerated by an electric field, through the insulating film, and an embrittlement region 104 is formed in a region at a predetermined depth from the surface of the single crystal semiconductor substrate 100.

The depth at which the embrittlement region 104 is formed can be adjusted by the accelerating energy and the incidence angle of ions used for irradiation. The acceleration energy can be adjusted by an acceleration voltage, dosage, or the like. The embrittlement region 104 is formed at the same depth or substantially the same depth as the average depth at which the ions have entered. The thickness of a semiconductor film to be separated from the single crystal semiconductor substrate 100 in a later step depends on the depth to which ions are introduced. The depth at which the embrittlement region 104 is formed may range from 10 nm to 500 nm, preferably from 50 nm to 200 nm.

As a gas used for the ion irradiation, a hydrogen gas, a rare gas or the like can be used, and in this embodiment mode, a hydrogen gas is preferably used. When a hydrogen gas is used in an ion doping method, ion species which are generated are H⁺, H₂⁺ and H₃⁺, and the single crystal semiconductor substrate 100 is preferably irradiated with the largest number of H₃⁺ among H⁺, H₂⁺ and H₃⁺. The addition efficiency of H₃⁺ ions is better than that of H⁺ ions or H₂⁺ ions; thus, reduction in irradiation time can be achieved. Further, a crack is easily generated in the embrittlement region in a later step.

After the embrittlement region 104 is formed, the insulating film is removed and an insulating film 102 is newly formed. At this time, the insulating film is removed because it is highly likely that the insulating film may be damaged by the ion irradiation. Note that the insulating film need not be removed in a case where damages to the insulating film are not problematic.

The insulating film 102 is a layer to form bonding when substrates are attached, and thus the surface of the insulating film 102 preferably has high planarity.

As the insulating film 102, for example, a single layer of a silicon oxide film, a silicon oxynitride film, a silicon nitride film, a silicon nitride oxide film, or the like, or a stacked layer thereof can be used. These films can be formed by a thermal oxidation method, a CVD method, a sputtering method, or the like. Further, in a case where the insulating film 102 is formed by a CVD method, a silicon oxide film which is manufactured by a CVD method using organosilane such as tetraethoxysilane (abbreviation: TEOS) (chemical formula: Si(OC₂H₅)₄) can be used as the insulating film 102. Furthermore, alternatively, an insulating film containing silicon as its main component, such as silicon carbide (SiC) film may be used.

Note that the term “silicon oxynitride film” refers to a film which has a larger number of oxygen atoms than that of nitrogen atoms and which contains oxygen, nitrogen, silicon, and hydrogen at concentrations ranging from 50 at. % to 70 at. %, 0.5 at. % to 15 at. %, 25 at. % to 35 at. %, and 0.1 at. % to 10 at. %, respectively. Further, the term “silicon nitride oxide film” refers to a film which has a larger number of nitrogen atoms than that of oxygen atoms and which contains oxygen, nitrogen, silicon, and hydrogen at concentrations ranging from 5 at. % to 30 at. %, 20 at. % to 50 at. %, 25 at. % to 35 at. %, and 15 at. % to 25 at. %, respectively. The aforementioned ranges are ranges for cases measured using Rutherford backscattering spectrometry (RBS) and hydrogen forward scattering (HFS). Note that percentages of nitrogen, oxygen, silicon, and hydrogen fall within the ranges given above, where the total number of atoms contained in the silicon oxynitride layer or the silicon nitride oxide layer is defined as 100 at. %. A case in which a silicon oxide film is used as the first insulating film 102 is described below.

Next, a base substrate 120 is prepared (see the phase (B-1) of FIG. 1).

As the base substrate 120, a substrate having an insulating surface can be used. The base substrate 120 preferably has a thermal expansion coefficient equal to or substantially equal to that of a single crystal semiconductor substrate. For example, the base substrate 120 preferably has a thermal expansion coefficient of 25×10⁻⁷/° C. to 50×10⁻⁷/° C. In addition, the base substrate 120 preferably has a strain point of 580° C. to 700° C., more preferably, 600° C. to 700° C.

For example, as the base substrate 120, a glass substrate can be used. As a glass substrate, a non-alkali glass substrate of aluminosilicate glass, aluminoborosilicate glass, barium borosilicate glass, or the like is preferably used. With use of a glass substrate which can have a large area and is inexpensive as the base substrate 120, the cost can be reduced more than in the case of using a silicon wafer as the base substrate 120.

Note that the base substrate 120 is not limited to a glass substrate and can be an insulating substrate made of an insulator, such as a ceramic substrate, a quartz substrate, or a sapphire substrate; a semiconductor substrate made of a semiconductor, such as silicon; or the like.

As the base substrate 120, a large-sized substrate of 300 mm×300 mm or larger is preferably used. As such a large-sized substrate, a mother glass substrate which is developed for manufacture of liquid crystal panels is preferred. Substrates with the following sizes are known as mother glass substrates, for example: 3rd generation (550 mm×650 mm), 3.5th generation (600 mm×720 mm), 4th generation (680 mm×880 mm or 730 mm×920 mm), 5th generation (1100 mm×1300 mm), 6th generation (1500 mm×1850 mm), 7th generation (1870 mm×2200 mm), 8th generation (2200 mm×2400 mm), and the like. With use of a large-sized mother glass substrate as the base substrate 120 for manufacture of a semiconductor substrate, the size of the semiconductor substrate can be increased.

Although not described in this embodiment mode, an insulating layer may be formed over a surface of the base substrate 120. By provision of the insulating layer, in a case where impurities (such as an alkaline metal or an alkaline earth metal) are contained in the base substrate 120, the impurities can be prevented from being diffused into the semiconductor layer. The insulating layer may have either a single-layer structure or a stacked structure. As a material used to form the insulating layer, silicon oxide, silicon nitride, silicon oxynitride, silicon nitride oxide or the like can be used.

Then, the single crystal semiconductor substrate 100 and the base substrate 120 are transported into a semiconductor substrate manufacturing apparatus, and a surface of each substrate is subjected to surface treatment (the phases (C-1) and (C-2) of FIG. 1). As the surface treatment, ozone treatment (e.g., cleaning with ozone water) or megasonic cleaning and cleaning with ozone water can be performed. Further, cleaning with ozone water and cleaning with hydrofluoric acid may be conducted repeatedly plural times. Through such surface treatment, dust such as an organic substance on each surface of the single crystal semiconductor substrate 100 and the base substrate 120 can be removed and each surface of the single crystal semiconductor substrate 100 and the base substrate 120 can be made hydrophilic. In addition, the surfaces of the substrates to be closely attached are cleaned sufficiently, so that the bonding strength can be increased.

Then, the base substrate 120 and the single crystal semiconductor substrate 100 described above are attached to each other (the phase (C-3) of FIG. 1). Specifically, it is preferable that the cleaned surface of the base substrate 120 is set to be in contact with the cleaned surface of the insulating film 102, and pressurizing treatment is conducted so that bonding is formed between the surface of the base substrate 120 and the surface of the insulating film 102. Hydrogen bonding and Van del Waals force seem to act on this formation of bonding. This bonding process of substrates can be performed at normal temperature (room temperature) without heat treatment; therefore, a substrate with low heat resistance, which has an allowable temperature limit of 700° C. or lower, like a glass substrate, can be used as the base substrate 120.

Next, the single crystal semiconductor substrate 100 attached to the base substrate 120 is irradiated with an electromagnetic wave 123 having a frequency of 300 MHz to 300 GHz (the phase (C-4) of FIG. 1).

When the single crystal semiconductor substrate 100 is irradiated with the electromagnetic wave 123 having a frequency of 300 MHz to 300 GHz, the single crystal semiconductor substrate 100 generates heat by the action of the electric field of the electromagnetic wave 123. Due to increase in temperature of the single crystal semiconductor substrates 100, the element or molecules added in the formation of the embrittlement region 104 separate out into microvoids that are formed in the embrittlement region 104, whereby internal pressure increases. Due to increase in pressure, change in the volume of the microvoids causes cracks in the embrittlement region 104. As a result, the single crystal semiconductor substrate 100 is separated along the embrittlement region 104 (the phase (C-5) of FIG. 1). Because the insulating film 102 is bonded to the base substrate 120, a single crystal semiconductor layer 124 which is separated from the single crystal semiconductor substrate 100 is fixed to the base substrate 120.

In this embodiment mode, the semiconductor substrate is separated by irradiation with an electromagnetic wave having a frequency of 300 MHz to 300 GHz. Thus, even when the temperature rise of the single crystal semiconductor substrate is 400° C. or lower, separation can be conducted with the embrittlement region as a separation plane, and irradiation with the electromagnetic wave can be completed in as short a time as five minutes or shorter. In addition, a glass substrate hardly absorbs electromagnetic waves in this frequency band. Accordingly, even when a glass substrate is used as the base substrate, the glass substrate is not heated to a temperature which may cause shrinkage of the glass substrate, and thus there is no need of intentional control of temperature of the glass substrate, such as cooling. Thus, shrinkage of the glass substrate can be suppressed.

Electromagnetic waves used for the electromagnetic wave irradiation process have a wavelength of 1 mm to 1 m and are in any of ultra high frequency (UHF), super high frequency (SHF), and extremely high frequency (EHF) bands. The frequency of each band is as follows.

UHF: 300 MHz to 3 GHz

SHF: 3 GHz to 30 GHz

EHF: 30 GHz to 300 GHz

In this embodiment mode, hydrogen is added to the single crystal semiconductor substrate 100 to form the embrittlement region, and thus the single crystal semiconductor layer 124 fixed to the base substrate 120 contains hydrogen. Preferably, the single crystal semiconductor layer 124 is heated at 410° C. or higher so that a hydrogen gas is released from the single crystal semiconductor layer 124 to reduce the concentration of hydrogen (the phase (C-6) of FIG. 1). In addition, the hydrogen concentration of the single crystal semiconductor layer 124 after the heat treatment is preferably 1×10²¹ atomic/cm³ or lower. By this heat treatment, the bonding strength of the bonding portion formed in fixing the substrates can be increased. The temperature of the heat treatment is preferably 500° C., more preferably 550° C.

Through the above process, a semiconductor substrate (SOT substrate) 135 can be manufactured, in which the single crystal semiconductor layer 124 is provided over the base substrate 120 with the insulating film 102 interposed therebetween.

Note that in the above process, planarization treatment may be performed on a surface of the obtained semiconductor substrate 135. By performing the planarization treatment, even when the single crystal semiconductor layer 124 provided over the base substrate 120 has an uneven surface after separation, the surface of the semiconductor substrate 135 can be planarized.

Planarization treatment can be performed using CMP (Chemical Mechanical Polishing) treatment, etching treatment, laser irradiation, or the like. For example, after either dry etching or wet etching or etching using a combination of both of them is performed (etch-back treatment), the single crystal semiconductor layer 124 is irradiated with laser light, whereby the single crystal semiconductor layer 124 can be recrystallized and planarized.

An upper surface of the single crystal semiconductor layer is irradiated with laser light so that it can be melted. After melting the upper surface of the single crystal semiconductor layer, the single crystal semiconductor layer is cooled down and solidified, whereby a single crystal semiconductor layer with improved upper surface planarity can be obtained. By using laser light, the base substrate 120 is not directly heated, and the temperature rise of the base substrate 120 can be suppressed. Therefore, a low heat-resistant substrate such as a glass substrate can be used as the base substrate 120.

As described above, when the embrittlement region is formed by addition of hydrogen to the single crystal semiconductor substrate, the single crystal semiconductor layer separated from the single crystal semiconductor substrate also contains a large amount of hydrogen. If hydrogen blows out of the single crystal semiconductor layer by irradiation of the single crystal semiconductor layer with laser light, crystallinity recovery and planarization cannot be achieved. If hydrogen concentration is higher than 1×10²¹ atomic/cm³, it is difficult to control energy density of laser light so as to achieve crystallinity recovery and planarization.

In the semiconductor substrate manufacturing apparatus described in this embodiment mode, heat treatment at 410° C. or higher is performed before laser irradiation treatment to reduce the hydrogen concentration of the single crystal semiconductor layer, whereby a hydrogen gas can be prevented from blowing out of the single crystal semiconductor layer due to laser irradiation (the phase (C-6) of FIG. 1). This makes it easy to control irradiation energy of laser light needed for crystallinity recovery and planarization. In other words, when heat treatment is performed in advance, the applicable irradiation energy range for the laser irradiation treatment can be widened, and crystallinity recovery and planarization can be performed with high reproducibility through the laser irradiation. In order to the ensure reproducibility of the effect of the laser irradiation treatment, the hydrogen concentration of the single crystal semiconductor layer is preferably made to be 1×10²¹ atomic/cm³ or less, more preferably, 7×10²⁰ atomic/cm³ or less through the heat treatment in the phase (C-6) of FIG. 1.

Note that it is preferable that the single crystal semiconductor layer be partially melted by the laser irradiation. This is because, if the single crystal semiconductor layer is completely melted, it is microcrystallized due to disordered nucleation after being in a liquid phase, so that crystallinity of the single crystal semiconductor layer is highly likely to decrease. On the contrary, by partial melting, crystal growth proceeds from a solid phase part, which is not melted. Accordingly, defects in the semiconductor layer can be reduced. Note that the term “complete melting” here refers to that the single crystal semiconductor layer is melted down to the vicinity of the lower interface of the single crystal semiconductor layer to be made in a liquid state. On the other hand, in this case, the term “partial melting” means that the upper part of the single crystal semiconductor layer is melted to be made in a liquid phase while the lower part thereof is not melted and is still in a solid phase.

A pulsed laser is preferably used for the laser irradiation. This is because high-energy pulsed laser light can be emitted instantaneously and the melting state can be easily obtained. The repetition rate is preferably from 1 Hz to 10 MHz.

After the above-described laser irradiation, a process of reducing the thickness of the single crystal semiconductor layer may be performed. In order to thin the single crystal semiconductor layer, one of dry etching and wet etching or a combination of both of the etchings may be employed (etch back treatment). For example, in the case where the single crystal semiconductor layer is a layer formed using a silicon material, the single crystal semiconductor layer can be thinned by dry etching treatment using SF₆ and O₂ as a process gas.

Note that planarization treatment may also be performed on the single crystal semiconductor substrate 100 which has been separated, in addition to the semiconductor substrate 135 (the phase (D-1) of FIG. 1). By planarizing the surface of the semiconductor substrate 100 which has been separated, the semiconductor substrate 100 can be reused in a process for manufacturing the semiconductor (SOI) substrate. The reprocessed single crystal semiconductor substrate is thinner at least by the thickness of the single crystal semiconductor layer 124 which has been separated.

FIGS. 2A and 2B illustrate a semiconductor substrate manufacturing apparatus of this embodiment mode. In FIGS. 2A and 2B, the semiconductor substrate manufacturing appratus includes a cleaning portion 200, an electromagnetic wave irradiation portion 202 and a heat treatment portion 204. In the semiconductor substrate manufacturing apparatus of this embodiment mode, the phases (C-1) to (C-6) of FIG. 1 can be conducted. A reprocessing portion for a single crystal semiconductor substrate to planarize the single crystal semiconductor substrate 100 after the single crystal semiconductor layer 124 is fixed to the base substrate 120 (a chamber for the phase (D-1) of FIG. 1) may be provided additionally. By provision of the reprocessing portion for a single crystal semiconductor substrate, throughput in manufacturing semiconductor substrates can be further increased. Hereinafter, a method for manufacturing a semiconductor substrate using the semiconductor substrate manufacturing apparatus illustrated in FIGS. 2A and 2B is described.

The single crystal semiconductor substrate 100 which has undergone the phase (A-1) of FIG. 1 and the base substrate 120 which has undergone the phase (B-1) of FIG. 1 are each transported to a cleaning portion 200 with use of a transfer robot 206. The cleaning portion 200 includes a washing tub 208, and the surface of the single crystal semiconductor substrate 100 or the base substrate 120 can be subjected to surface treatment with use of the washing tub 208 as illustrated in the phase (C-1) or (C-2) of FIG. 1. In addition, although one washing tub 208 is provided in the example of FIGS. 2A and 2B, the single crystal semiconductor substrate 100 and the base substrate 120 are preferably washed in different washing tubs 208 in order to increase the throughput.

The single crystal semiconductor substrate 100 and the base substrate 120 which have been subjected to surface treatment are transported to the electromagnetic wave irradiation portion 202 by a transfer robot 206. The single crystal semiconductor substrate 100 and the base substrate 120 which have been transported to the electromagnetic wave irradiation portion 202 are attached to each other in the electromagnetic wave irradiation portion 202 and then are set on a stage 218 which can be lifted up and down.

In the attachment of the single crystal semiconductor substrate 100 and the base substrate 120, for example, an arm 230 on which the single crystal semiconductor substrate 100 is set turns 180° around with the single crystal semiconductor substrate 100 sucked thereon, so as to be close to an arm 240 on which the base substrate 120 is set, whereby the single crystal semiconductor substrate 100 and the base substrate 120 can be attached to each other. Before the attachment of the substrates, alignment of the arm 230 and the arm 240 should be performed. In addition, after the attachment, suction by the arm 230 is stopped, and the single crystal semiconductor substrate 100 and the base substrate 120, which are bonded to each other, are set on the stage 218 by the arm 240.

The stage 218 on which the single crystal semiconductor substrate 100 and the base substrate 120 bonded to each other are set is lifted up so that the single crystal semiconductor substrate 100 can be close to a dielectric 216 provided in the upper portion of the electromagnetic wave irradiation portion (FIG. 2B). An electromagnetic wave output from an electromagnetic wave generator 210 provided outside the chamber of the electromagnetic wave irradiation portion 202, is transmitted to the dielectric 216 from slots 214 via waveguides 212. Accordingly, by bringing the dielectric 216 and the single crystal semiconductor substrate 100 into close contact with each other, as illustrated in the phase (C-4) of FIG. 1, the single crystal semiconductor substrate 100 can be heated by the electromagnetic wave transmitted to the dielectric 216. Further, as illustrated in the phase (C-5) of FIG. 1, by this electromagnetic wave irradiation process, a crack is generated in the embrittlement region formed in the single crystal semiconductor substrate 100 and the single crystal semiconductor substrate 100 is separated along the embrittlement region. The single crystal semiconductor layer 124, which has been separated from the single crystal semiconductor substrate 100, is fixed to the base substrate 120.

As the electromagnetic wave generator 210, an apparatus which can generate an electromagnetic wave having a frequency of 300 MHz to 300 GHz can be used, and for example, a microwave heating apparatus or a millimeter wave heating apparatus can be used. The microwave heating apparatus is an apparatus which heats an object by microwave radiation, and a millimeter wave heating apparatus is an apparatus which heats an object by millimeter wave radiation. Irradiation with the electromagnetic waves is performed in as a short time as five minutes or shorter, and can be performed in the range of one minute to three minutes.

When the stage 218 is lifted up or down, a pin 224 is preferably provided to prevent slipping or dropping of the single crystal semiconductor substrate 100 and the base substrate 120 set on the stage 218. Alternatively, the stage 218 may have a hollow portion and the single crystal semiconductor substrate 100 and the base substrate 120 bonded to each other may be set in the hollow portion.

The single crystal semiconductor substrate 100 from which the single crystal semiconductor layer 124 has been separated is collected in a cassette (not illustrated) for collecting the single crystal semiconductor substrate. In addition, the semiconductor substrate 135 including the fixed single crystal semiconductor layer 124 is transported to the heat treatment portion 204 by the transfer robot 206.

In the heat treatment portion 204, the heat treatment illustrated in the phase (C-6) of FIG. 1 is conducted and the hydrogen concentration of the single crystal semiconductor layer 124 is reduced. For the heat treatment, a diffusion furnace, a heating furnace such as a resistance heating furnace, rapid thermal annealing (RTA), or the like can be used. The temperature of the heat treatment is preferably 500° C. or higher, more preferably 550° C. or higher. When this heat treatment is performed with a heating furnace, for example, after the base substrate 120 provided with the single crystal semiconductor layer 124 is heated at a treatment temperature of 500° C. for one hour, the heating temperature may be raised to 550° C., at which heating may be performed for four hours. This heat treatment can be performed not for the purpose of decreasing the hydrogen concentration of the single crystal semiconductor layer 124 but for the purpose of increasing bonding force in the bonding portion formed through the substrate fixing treatment.

In this embodiment mode, the heat treatment portion 204 includes an RTA chamber 220 and a slow-cooling chamber 222. In the RTA chamber 220, the semiconductor substrate 135 which has undergone the heat treatment illustrated in the phase (C-6) of FIG. 1 is slowly cooled down in the slow-cooling chamber 222 and then is used in a device process.

After the heat treatment, the single crystal semiconductor layer 124 may be subjected to a planarization process. When a laser irradiation process is conducted as the planarization process, hydrogen concentration of the single crystal semiconductor layer is reduced in the heat treatment portion 204, so that a hydrogen gas can be prevented from blowing out of the single crystal semiconductor layer due to the laser irradiation. Therefore, irradiation energy of laser light needed for crystallinity recovery and planarization can be easily controlled.

As for the semiconductor substrate manufacturing apparatus in this embodiment mode, a cleaning portion for a bonding surface and an electromagnetic wave irradiation portion for attachment of cleaned substrates are included in the same apparatus, a step of transferring substrates having a treated surface to another appratus can be omitted, and thus attachment of dusts generated in transferring cleaned substrates can be suppressed. Therefore, the surfaces (bonding surfaces) of the single crystal semiconductor substrate and the base substrate can be kept sufficiently clean, and thus defective attachment due to contamination can be suppressed.

In addition, after the base substrate and the single crystal semiconductor substrate are attached to each other, heat treatment is conducted using electromagnetic wave irradiation in the same chamber. Thus, the base substrate can be prevented from sagging when it is transported. Accordingly, accurate attachment can be performed even when the size of the base substrate is increased.

FIGS. 3A to 3C illustrate a typical structure of a main portion of the electromagnetic wave irradiation portion included in the semiconductor substrate manufacturing appratus in this embodiment mode. FIG. 3A is a side view of the electromagnetic wave irradiation portion 202 illustrated in FIGS. 2A and 2B, and FIG. 3B is a top view of the electromagnetic wave irradiation portion 202.

As illustrated in FIGS. 3A and 3B, the electromagnetic wave irradiation portion 202 includes a plurality of waveguides 212 which are arranged in parallel, in a chamber 248, and the waveguides 212 are supported by a cover plate 242 and a bottom plate 244. The cover plate 242 and the bottom plate 244 are preferably formed using the same material and unified. As a material for the cover plate 242 and the bottom plate 244, a conductive material, e.g., aluminum can be used. Although not illustrated, an end portion of the waveguide 212 is connected to an electromagnetic wave generator outside the chamber 248. In addition, the number of electromagnetic wave generator is not limited to one, and a plurality of electromagnetic wave generators may be provided. If a plurality of electromagnetic wave generators are provided, the plurality of electromagnetic wave generators preferably generate electromagnetic waves having the same frequency.

In the electromagnetic wave irradiation portion 202, the stage 218 for setting the single crystal semiconductor substrate 100 and the base substrate 120, which are bonded to each other, is provided. The stage 218 is formed using a ceramic material such as aluminum nitride, silicon nitride, or silicon carbide, and the stage 218 can be lifted up and down. The single crystal semiconductor substrate 100 and the base substrate 120 set on the stage 218 are lifted up and down together with the stage 218, and thus the height for lifting up the single crystal semiconductor substrate 100 and the base substrate 120 by the stage 218 in the electromagnetic wave irradiation portion 202 can be controlled.

The plurality of waveguides 212 each have a plurality of slots 214 as through holes. In addition, on the lower side of each slot 214, a dielectric 216 is provided. The dielectrics 216 are attached to the bottom plate 244 with use of an attachment 246 formed from the same material as the bottom plate 244. In this embodiment mode, the slots 214 having a slit shape are formed in the bottom plate 244. The interval between slots 214 is preferably half of a wavelength which is transmitted in the waveguide 212. Note that the shape of the slot 214 is not limited to the slit shape, and a wide variety of shapes can be employed for the slots. Further, the bottom plate 244 and the slots 214, and the slots 214 and the dielectrics 216 are adhered to each other by a sealing member such as an O ring.

The dielectrics 216 are each formed using ceramics such as sapphire, quartz glass, alumina, silicon oxide, or silicon nitride, and are provided in close contact with the slots 214. An electromagnetic wave generated by the electromagnetic wave generator is transmitted to the dielectrics 216 via the waveguides 212 and the slots 214, and is released into the process container through the dielectrics 216.

In the semiconductor substrate manufacturing appratus in this embodiment mode, the plurality of dielectrics 216 are provided for the electromagnetic wave irradiation portion, whereby a large area can be uniformly irradiated with an electromagnetic wave. Thus, in a case where a single crystal semiconductor layer formed over a substrate having one side of 600 mm or longer, in particular, over a large area substrate having one side of 1000 mm, is heated, the area can be uniformly heated. In addition, the size and weight of each dielectric 216 can be reduced, and thus the electromagnetic wave irradiation portion can be manufactured at low cost.

With use of the above-described semiconductor substrate manufacturing appratus in this embodiment mode, a semiconductor substrate having a single crystal semiconductor layer on its insulating surface can be manufactured with high throughput.

Note that FIGS. 3A to 3C illustrate an example in which one single crystal semiconductor substrate 100 is bonded to the base substrate 120, but this example of the manufacturing method of a semiconductor substrate is a non-limiting example. A plurality of single crystal semiconductor substrates 100 may bonded to the base substrate 120. In a case where the plurality of single crystal semiconductor substrates 100 are bonded to the base substrate 120, the plurality of single crystal semiconductor substrates 100 may be bonded all at once, or they may be bonded over plural times.

The semiconductor substrate manufacturing appratus in this embodiment mode makes it possible to uniformly irradiate a large area with an electromagnetic wave. For example, even in a case where a plurality of single crystal semiconductor substrates 100 are attached to a large base substrate 120 all at once, and then separated therefrom, uniform heating is possible. Further, even in a case where a glass substrate is used as the base substrate 120, the glass substrate is not heated to a temperature at which shrinkage may occur, and thus there is no need for intentionally temperature controlling of the glass substrate, and shrinkage of the glass substrate can be suppressed. Accordingly, even in a case where a step of attaching a single crystal semiconductor substrate (a substrate fixing process) and a step of separating the single crystal semiconductor substrate (an electromagnetic wave irradiation process) are conducted repeatedly twice or more to a large-size glass substrate, a plurality of single crystal semiconductor layers can be attached to the glass substrate with high accuracy. In particular, in a case where a plurality of single crystal semiconductor substrates 100 are attached over plural times, while the first bonding and separation is conducted in the electromagnetic wave irradiation portion, the single crystal semiconductor substrate to be used for the second bonding and separation is cleaned in a cleaning portion, whereby a plurality of single crystal semiconductor substrates can be attached to the large base substrate 120 at high throughput.

In addition, in the semiconductor substrate manufacturing apparatus in this embodiment mode, a structure of the electromagnetic wave irradiation portion is not limited to the structure illustrated in FIGS. 3A and 3B. For example, a port for controlling the atmosphere in the chamber 248 may be provided, or a mechanism for heating the stage may be provided. As illustrated in FIG. 3C, a structure may be employed, in which the structure of FIG. 3A is inverted and the waveguides 212 are provided on the lower side of the chamber and the single crystal semiconductor substrate 100 and the base substrate 120 are directly set on the dielectrics 216 provided above the waveguides 212. By provision of the single crystal semiconductor substrate 100 and the base substrate 120 directly on the dielectrics 216, setting of the stage is not needed in the electromagnetic wave irradiation portion, so that the appratus can be downsized.

In FIGS. 3A to 3C, the single crystal semiconductor substrate 100 and the dielectrics 216 face each other; however, the base substrate 120 and the dielectrics 216 may be made to face each other, because an electromagnetic wave having a frequency of 300 MHz to 300 GHz is hardly absorbed by a glass substrate.

The method for manufacturing a semiconductor substrate described in this embodiment mode can be combined with a manufacturing method described in any of the other embodiment modes in this specification, as appropriate.

### Embodiment Mode 2

In Embodiment Mode 2, a structure of a semiconductor substrate manufacturing appratus will now be described, which is different from the structure described in Embodiment Mode 1. Specifically, another structure of an electromagnetic wave irradiation portion which is different from that illustrated in FIGS. 3A to 3C will be described.

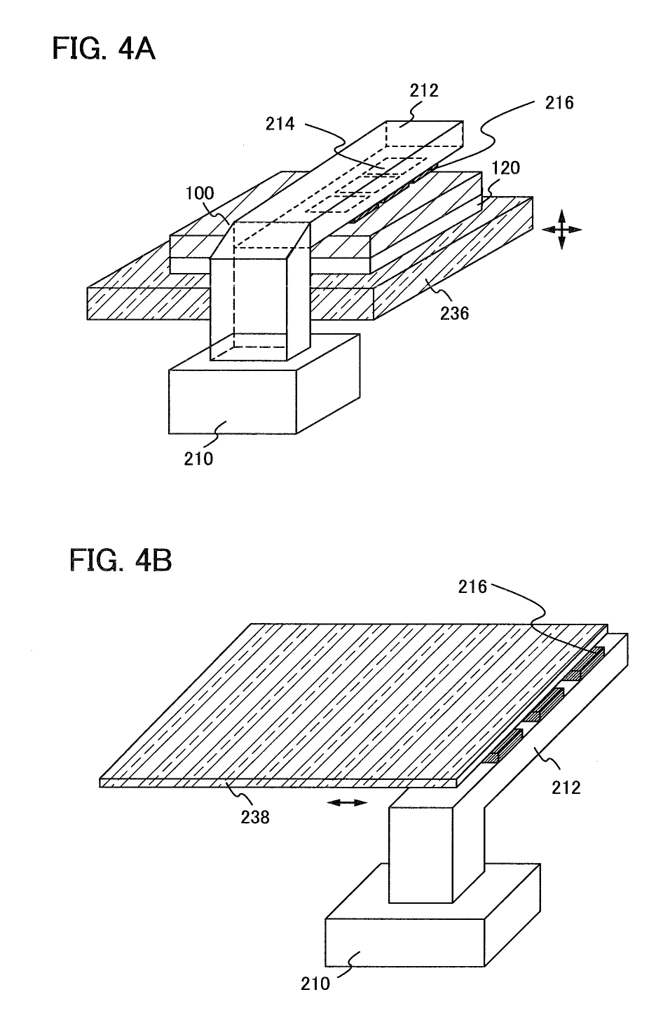

FIG. 4A is a cross-sectional view of a main portion of an electromagnetic wave irradiation portion. In FIGS. 4A and 4B, drawing of a chamber is omitted. In this embodiment mode, a waveguide 212 including a plurality of slots 214 as through holes is provided for the electromagnetic wave irradiation portion. An end portion of the waveguide 212 is connected to an electromagnetic wave generator 210 provided outside the electromagnetic wave irradiation portion. The waveguide 212 is supported by a cover plate and a bottom plate. The top body and the bottom plate are preferably formed using the same material and unified.

A dielectric 216 is attached to the lower side of each of the slots 214 with use of an attachment formed from the same material as the cover plate and the bottom plate. For the structure and material of the slot 214 and the dielectric 216, description of Embodiment Mode 1 can be referred to, and the description is omitted here.

The electromagnetic wave irradiation portion in this embodiment mode has a stage 236 which is movable in a vertical direction and a plane (horizontal) direction. The single crystal semiconductor substrate 100 and the base substrate 120, which are bonded to each other, are set on the stage 236 and then lifted up so as to be close to the dielectrics 216. After that, the single crystal semiconductor substrate 100 and the base substrate 120 are irradiated with an electromagnetic wave having a frequency of 300 MHz to 300 GHz via the dielectrics 216 and the stage 236 is moved in the plane direction. Therefore, without providing a plurality of waveguides 212, the entire surface of the single crystal semiconductor substrate 100 set on the stage 236 can be irradiated with an electromagnetic wave, and thus can be uniformly heated. By this heat treatment, a single crystal semiconductor layer can be fixed to the base substrate 120 by using an embrittlement region formed in the single crystal semiconductor substrate 100 as a separation plane.

In the semiconductor substrate manufacturing appratus described in this embodiment mode, the number of slots can be reduced as compared with the case where the plurality of waveguides are provided. Therefore, leakage of the electromagnetic wave from the slots can be reduced, and the electromagnetic wave generated in the electromagnetic wave generator 210 can be used efficiently. Further, since the plurality of waveguides 212 are not provided, the structure of the appratus is simplified, and thus the semiconductor substrate manufacturing appratus can be manufactured at low cost.

Note that FIG. 4A illustrates the structure in which electromagnetic wave irradiation is performed from the top surface side of the single crystal semiconductor substrate 100; however, the structure of the electromagnetic wave irradiation portion is not limited to this structure of FIG. 4A. For example, as illustrated in FIG. 4B, a structure may be employed, in which a plurality of slots are formed on the top surface of the waveguide 212, and the dielectric 216 is formed above each slot, and the stage 238 movable in a plane direction is directly provided over the dielectric 216. The single crystal semiconductor substrate 100 and the base substrate 120, which are bonded to each other, are set on the stage 238, and are irradiated with an electromagnetic wave through the dielectrics 216 from the bottom side of the stage 238, and the stage 238 is moved. In this manner, the entire surface of the single crystal semiconductor substrate 100 set on the stage 238 can be irradiated with an electromagnetic wave and uniformly heated. With use of the structure illustrated in FIG. 4B, because the stage 238 can be formed directly on the dielectrics 216, a lifting function of the stage 238 can be omitted. Accordingly, the appratus illustrated in FIG. 4B is more simplified than the structure illustrated in FIG. 4A, and thus the semiconductor substrate manufacturing apparatus can be downsized and manufactured at low cost.

The process for separating the single crystal semiconductor substrate 100 is conducted by irradiation with an electromagnetic wave having a frequency of 300 MHz to 300 GHz. Thus, when the temperature rise of the single crystal semiconductor substrate is 400° C. or lower, the single crystal semiconductor substrate can be separated with the embrittlement region as a separation plane. Therefore, in the process for separating the single crystal semiconductor substrate 100, the temperature rise of the base substrate can also be 400° C. or lower, and thus, even when a glass substrate is used as the base substrate, shrinkage of the glass substrate can be suppressed. In this manner, a plurality of single crystal semiconductor layers 124 can be fixed to the base substrate 120, i.e., a substrate which easily shrinks, such as a glass substrate, by plural times of fixing process and separation process for the single crystal semiconductor substrate. Moreover, a plurality of single crystal semiconductor layers can be fixed to a substrate which easily shrinks, with high positional accuracy.

In addition, the temperature rising of the base substrate 120 and the single crystal semiconductor substrate 100 can be suppressed within 400° C. or lower in the electromagnetic wave irradiation process. Thus, in the process for separating the single crystal semiconductor substrate 100, the single crystal semiconductor substrate 100 can be prevented from being broken due to a difference in thermal expansion from the base substrate 120. Therefore, as the base substrate, a substrate, e.g., a quartz substrate, having a greatly different thermal expansion coefficient from that of the single crystal semiconductor substrate 100 (specifically, a single crystal silicon substrate) can be used (for example, a substrate having a thermal expansion coefficient which is five times or more as high as that of the single crystal semiconductor substrate 100 can be used).

The method for manufacturing a semiconductor substrate described in this embodiment mode can be combined with a manufacturing method described in any of the other embodiment modes in this specification, as appropriate.

### Embodiment Mode 3

In Embodiment Mode 3, a structure of another electromagnetic wave irradiation portion which is different from those of the electromagnetic wave irradiation portions in the above-described embodiment modes will now be described.

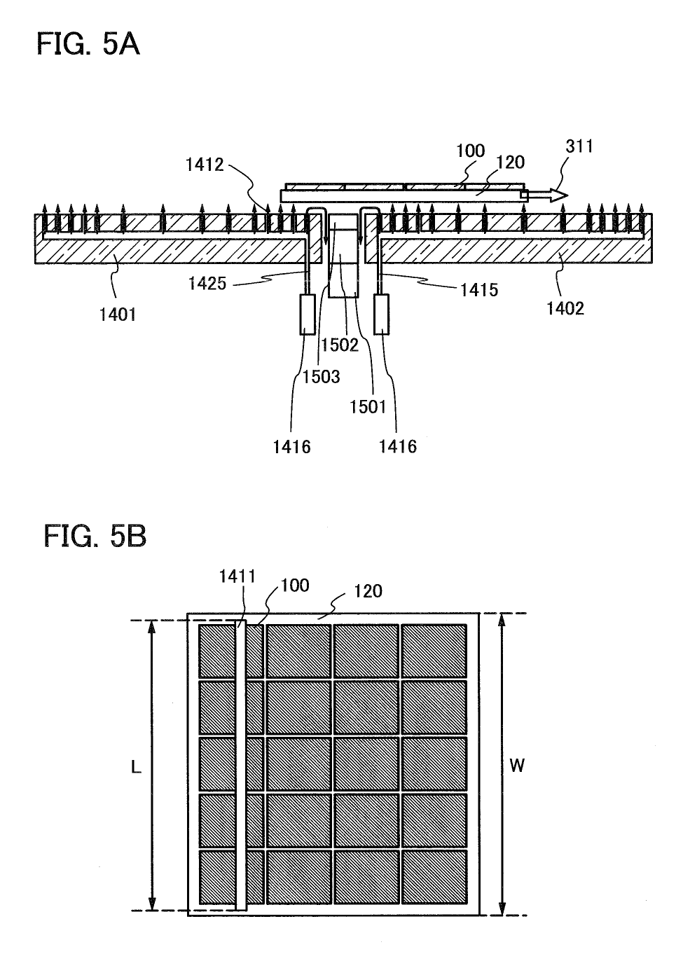

FIGS. 5A and 5B illustrate a structure of an electromagnetic wave irradiation portion in this embodiment mode. FIG. 5A is a cross-sectional view of a main portion of the electromagnetic wave irradiation portion. This electromagnetic wave irradiation portion includes a plurality of stages in a chamber, and a mechanism by which a gas such as air is blown from gas blowing holes provided in the stages in order to float a substrate and transfer it. In FIGS. 5A and 5B, drawing of the chamber is omitted.

In a case where a large area base substrate 120 is used, preferably, an area to be irradiated with an electromagnetic wave from a waveguide, i.e., the length L of an area in which slots are formed is increased and the large area base substrate 120 is moved in one direction so that the processing time for electromagnetic wave irradiation can be shortened.

FIG. 5B is a top view illustrating a positional relationship among the base substrate 120, an area 1411 irradiated with the electromagnetic wave (irradiation area 1411) and the single crystal semiconductor substrate 100 in the electromagnetic wave irradiation. As illustrated in FIG. 5B, the length L of the irradiation area 1411 is greater than the total length of five single crystal semiconductor substrates 100 that are aligned. The width of the base substrate 120 is denoted by W. Note that the length of the irradiation area 1411 in a direction perpendicular to the width W of the base substrate 120 is referred to as a width of the electromagnetic wave. Here, an example is given in which the base substrate 120 has a size of 600 mm×720 mm and twenty single crystal semiconductor substrates 100 are arranged over one base substrate 120.

In the electromagnetic wave irradiation portion of this embodiment mode, a waveguide 1502 and a dielectric 1503 are provided to transmit an electromagnetic wave from an electromagnetic wave generator 1501 provided outside the electromagnetic wave irradiation portion (FIG. 5A). The frequency of the electromagnetic wave may be in the range of 300 MHz to 300 GHz, and for example, a frequency of 2.45 GHz can be given. The surface of the dielectric 1503 is on an extension of the surface of the stage 1401, and also serves as a stage. Note that for the structures of the waveguide 1502 and the dielectric 1503, the description of Embodiment Mode 2 with reference to FIGS. 4A and 4B can be referred to, and thus the description about the waveguide 1502 and the dielectric 1503 is omitted here.

A gas (e.g., air in this embodiment mode) is supplied from a gas blower 1416 to a plurality of gas blowing holes 1412 of the stage 1401 through a tube 1425. In the supply of air, the amount and pressure of air is controlled by the gas blower 1416 so that the base substrate 120 can be floated.

The stage 1402 is also provided with a plurality of gas blowing holes 1412. Air is supplied from the gas blower 1416 to the plurality of gas blowing holes 1412 of the stage 1402 through the tube 1415.

Note that although the gas blowers are illustrated below the stages in FIG. 5A, the structure is merely an example for description and there is no particular limitation on the location of the gas blowers. It is needless to say that the gas blowers can be placed in another position by increasing the length of each tube.

With air blown from the plurality of gas blowing holes 1412 provided in the two stages 1401 and 1402, the substrate can be floated, and force is added to the transfer direction by a transfer roller (not shown) arranged in both sides, whereby the base substrate 120 can be transported in a direction indicated by an arrow 311. Thus, the entire surface of the single crystal semiconductor substrate 100 bonded to the base substrate 120 can be irradiated with an electromagnetic wave having a frequency of 300 MHz to 300 GHz, and uniformly heated. By this heating, a single crystal semiconductor layer can be fixed to the base substrate 120 by using an embrittlement region formed in the single crystal semiconductor substrate 100 as a separation plane. A lager number of gas blowing holes are provided in an end portion of the stage than in a central portion, thereby preventing the substrate from sagging.

In this embodiment mode, the plurality of single crystal semiconductor substrates 100 are attached to the base substrate 120, but the orientations of main surfaces thereof may be the same or different. For example, when a single crystal semiconductor substrate whose crystal structure is a diamond structure, such as a single crystal silicon substrate, is used as the single crystal semiconductor substrate 100, the plane orientation of its main surface may be (100), (110), or (111). In addition, the plurality of single crystal semiconductor substrates 100 attached to one base substrate 120 may have the same or different electric characteristics such as conductivity (n type, i type or p type), resistance and the like. Further, either when one single crystal semiconductor substrate is attached to the base substrate, or when a plurality of single crystal semiconductor substrates are attached to the base substrate over plural times, a semiconductor substrate manufacturing apparatus described in this embodiment mode can be used.

In the semiconductor substrate manufacturing apparatus described in this embodiment mode, a substrate is floated and transported; thus the entire surface of the single crystal semiconductor substrate 100 bonded to the base substrate 120 can be irradiated with an electromagnetic wave, and uniformly heated. By this heating, a single crystal semiconductor layer can be fixed to the base substrate 120 by using an embrittlement region formed in the single crystal semiconductor substrate 100 as a separation plane.

The length L of the area irradiated with an electromagnetic wave (the irradiation area 1411) from the waveguide, i.e., an area in which slots are formed is increased and the large area base substrate 120 is moved in one direction, so that electromagnetic wave irradiation can be completed. Thus, processing time of the electromagnetic wave irradiation process can be shortened.

The process for separating the single crystal semiconductor substrate 100 is conducted by irradiation with an electromagnetic wave having a frequency of 300 MHz to 300 GHz. Thus, even when the range of temperature rise of the single crystal semiconductor substrate is 400° C. or lower, the single crystal semiconductor substrate can be separated by using the embrittlement region as a separation plane. Therefore, the process for separating the single crystal semiconductor substrate 100, the temperature rise of the base substrate can also be 400° C. or lower, and thus, even when a glass substrate is used as the base substrate, shrinkage of the glass substrate can be suppressed. In this manner, a plurality of single crystal semiconductor layers 124 can be fixed to the base substrate 120, i.e., a substrate which easily shrinks, such as a glass substrate, by plural times of fixing process and separation process for the single crystal semiconductor substrate. Moreover, a plurality of single crystal semiconductor layers can be fixed to a substrate which easily shrinks, with high positional accuracy.

In addition, the temperature rise of the base substrate 120 and the single crystal semiconductor substrate 100 can be 400° C. or lower in the electromagnetic wave irradiation process. Thus, in the process for separating the single crystal semiconductor substrate 100, the single crystal semiconductor substrate 100 can be prevented from being broken due to a difference in thermal expansion coefficient from the base substrate 120. Therefore, as the base substrate, a substrate formed with a different material, e.g., a quartz substrate, having a greatly different thermal expansion coefficient from that of the single crystal semiconductor substrate 100 (specifically, a single crystal silicon substrate) can be used (for example, a substrate having a thermal expansion coefficient which is five times or more as high as that of the single crystal semiconductor substrate 100 can be used).

The method for manufacturing a semiconductor substrate described in this embodiment mode can be combined with a manufacturing method described in any of the embodiment modes in this specification, as appropriate.

### Embodiment Mode 4

In Embodiment Mode 4, a method for manufacturing a semiconductor device with use of a semiconductor substrate which is manufactured by using a semiconductor substrate manufacturing appratus will be described.

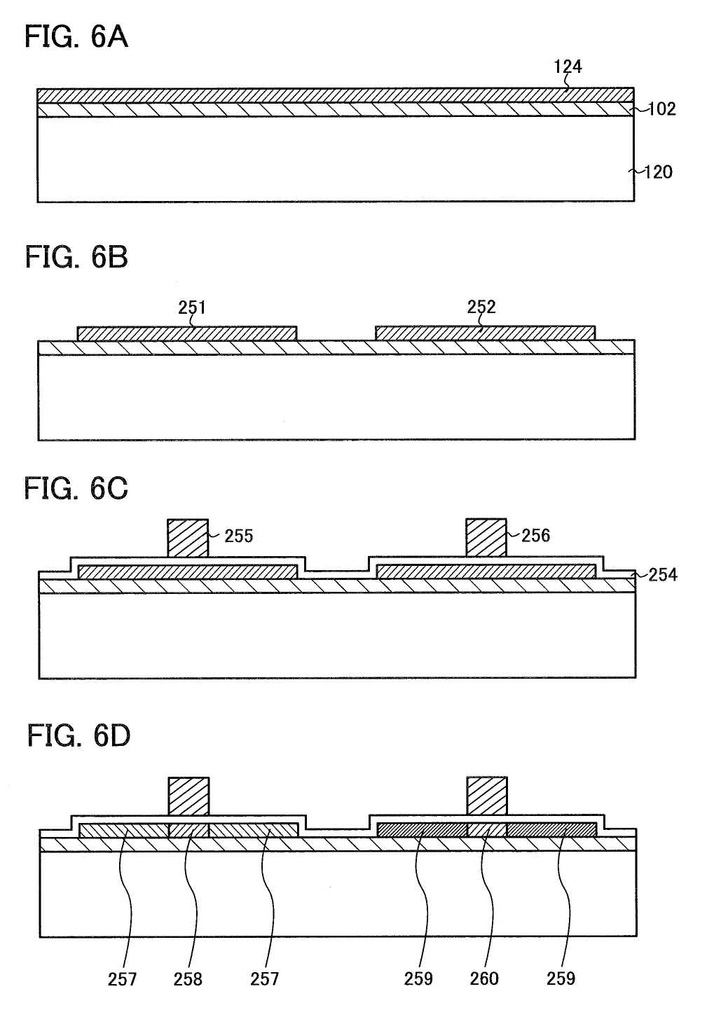

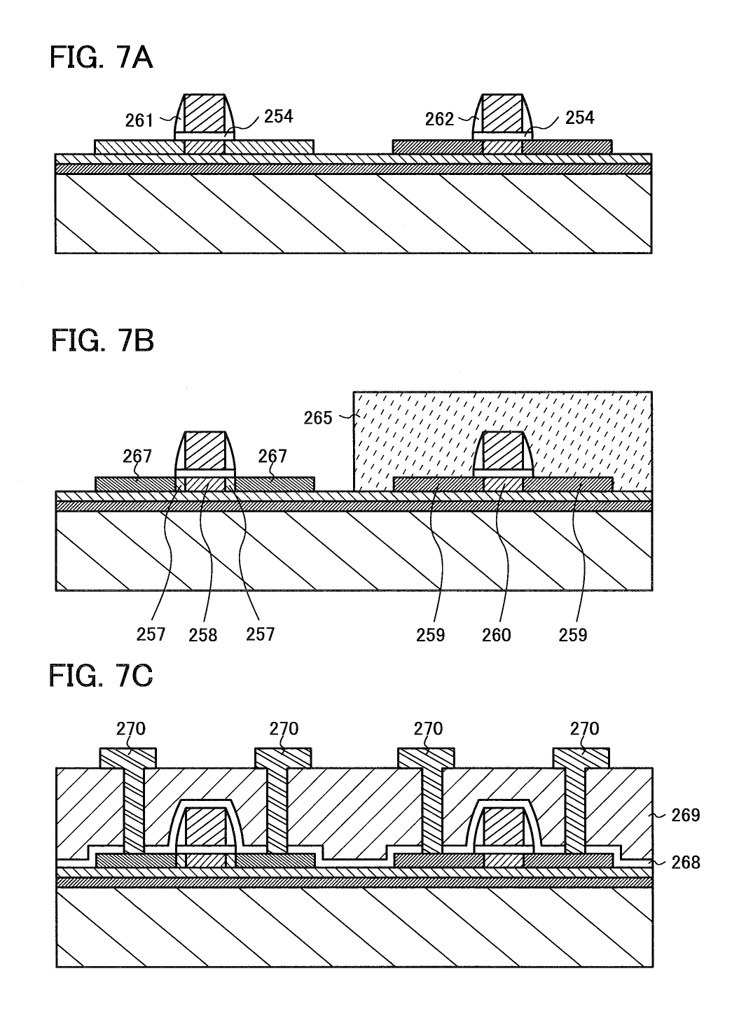

First, a method for manufacturing an n-channel thin film transistor and a p-channel thin film transistor will be described as a method for manufacturing a semiconductor device with reference to FIGS. 6A to 6D and FIGS. 7A to 7C. By combination of a plurality of thin film transistors (TFTs), a variety of semiconductor devices can be manufactured.

FIG. 6A is a cross-sectional view of a semiconductor substrate which is manufactured by using the semiconductor substrate manufacturing appratus in any of the above-described embodiment modes.

The single crystal semiconductor layer 124 is separated into each element by etching to form semiconductor films 251 and 252 as illustrated in FIG. 6B. The semiconductor film 251 is used for an n-channel TFT, and the semiconductor film 252 is used for a p-channel TFT.

As illustrated in FIG. 6C, an insulating film 254 is formed over the semiconductor films 251 and 252. Then, a gate electrode 255 is formed over the semiconductor film 251 with the insulating film 254 interposed therebetween, and a gate electrode 256 is formed over the semiconductor film 252 with the insulating film 254 interposed therebetween.

Before the single crystal semiconductor layer 124 is etched, it is preferable to add an impurity element which serves as an acceptor, such as boron, aluminum, or gallium, or an impurity element which serves as a donor, such as phosphorus or arsenic, into the single crystal semiconductor layer 124 in order to control the threshold voltage of the TFTs. For example, an acceptor is added into a region where the n-channel TFT is formed, and a donor is added into a region where the p-channel TFT is formed.

Next, as illustrated in FIG. 6D, n-type low-concentration impurity regions 257 are formed in the semiconductor film 251, and p-type high-concentration impurity regions 259 are formed in the semiconductor film 252. First, the n-type low-concentration impurity regions 257 are formed in the semiconductor film 251. In order to form the n-type low-concentration impurity regions 257, the semiconductor film 252 for the p-channel TFT is covered with a resist mask, and a donor is added into the semiconductor film 251. As the donor, phosphorus or arsenic may be added. When the donor is added by an ion doping method or an ion implantation method, the gate electrode 255 serves as a mask, and the n-type low-concentration impurity regions 257 are formed in the semiconductor film 251 in a self-aligned manner. A region of the semiconductor film 251 which overlaps with the gate electrode 255 serves as a channel formation region 258.

Next, after the mask which covers the semiconductor film 252 is removed, the semiconductor film 251 for the n-channel TFT is covered with a resist mask. Then, an acceptor is added into the semiconductor film 252 by an ion doping method or an ion implantation method. Boron can be added as the acceptor. At the step of adding the acceptor, the gate electrode 255 serves as a mask and the p-type high-concentration impurity regions 259 are formed in the semiconductor film 252 in a self-aligned manner. The p-type high-concentration impurity regions 259 serve as a source region and a drain region. A region of the semiconductor film 252 which overlaps with the gate electrode 256 serves as a channel formation region 260. Here, the method is described, in which the p-type high-concentration impurity regions 259 are formed after the n-type low-concentration impurity regions 257 are formed; however, the p-type high-concentration impurity regions 259 can be formed first.

Next, after the resist which covers the semiconductor film 251 is removed, an insulating film having a single layer structure of a nitrogen compound such as silicon nitride or an oxide such as silicon oxide or a stacked layer structure thereof is formed by a plasma CVD method or the like. This insulating film is anisotropically etched in a perpendicular direction to form sidewall insulating films 261 and 262 which are in contact with side surfaces of the gate electrodes 255 and 256, as illustrated in FIG. 7A. By this anisotropic etching, the insulating film 254 is also etched.

Next, as illustrated in FIG. 7B, the semiconductor film 252 is covered with a resist 265. In order to form high-concentration impurity regions serving as a source region and a drain region in the semiconductor film 251, donors are added into the semiconductor film 251 at a high dose by an ion implantation method or an ion doping method. The gate electrode 255 and the sidewall insulating film 261 serve as masks, and n-type high-concentration impurity regions 267 are formed. Then, heat treatment for activating the donors and the acceptors is performed.

After the heat treatment for activation, an insulating film 268 containing hydrogen is formed as illustrated in FIG. 7C. After the insulating film 268 is formed, heat treatment is performed at a temperature of from 350° C. to 450° C., hydrogen contained in the insulating film 268 is diffused into the semiconductor films 251 and 252. The insulating film 268 can be formed by deposition of silicon nitride or silicon nitride oxide by a plasma CVD method at a process temperature of 350° C. or lower. The supply of hydrogen to the semiconductor films 251 and 252 makes it possible to efficiently compensate defects which are to be trapping centers in the semiconductor films 251 and 252 and at an interface with the insulating film 254.

After that, an interlayer insulating film 269 is formed. The interlayer insulating film 269 can be formed with a film having a single layer structure or a stacked layer structure of any one or more of films selected from an insulating film containing an inorganic material, such as a silicon oxide film or a BPSG (borophosphosilicate glass) film, and an organic resin film containing polyimide, acrylic, or the like. After contact holes are formed in the interlayer insulating film 269, wirings 270 are formed as illustrated in FIG. 7C. The wirings 270 can be formed with a conductive film having a three-layer structure in which a low-resistance metal film such as an aluminum film or an aluminum-alloy film is sandwiched between barrier metal films. The barrier metal films can be formed using metal films containing molybdenum, chromium, titanium, or the like.

Through the above-described steps, a semiconductor device having the n-channel TFT and the p-channel TFT can be manufactured.

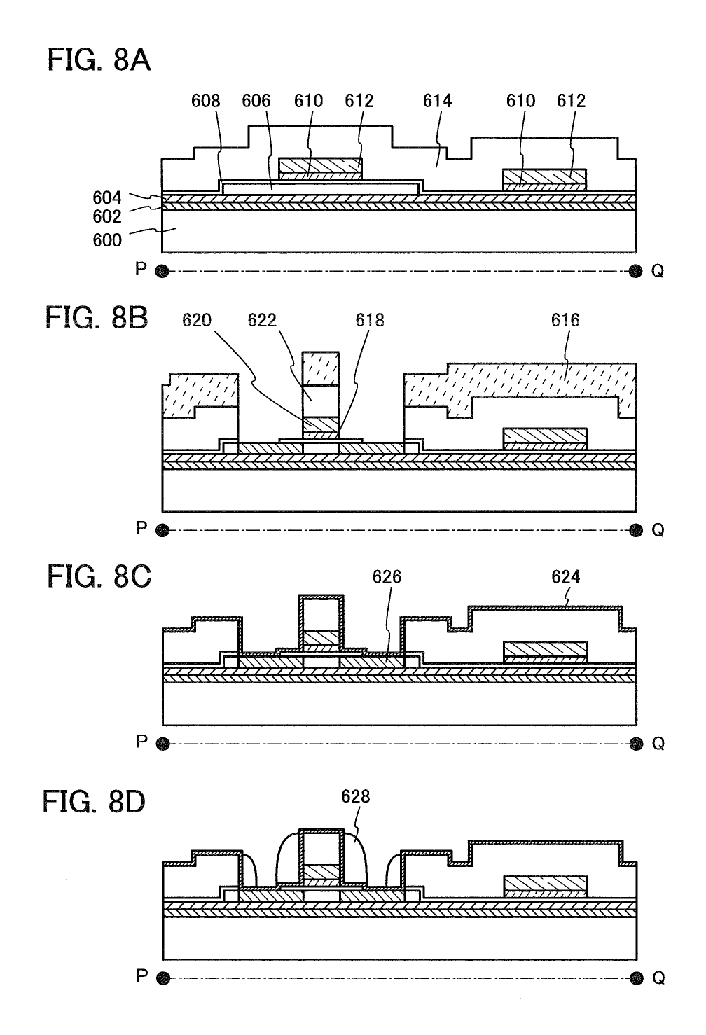

Moreover, another example of a manufacturing method of a thin film transistor which can be used for a semiconductor device will be described with reference to FIGS. 8A to 8D, FIGS. 9A to 9C and FIGS. 10A to 10D. Note that in a manufacturing method of a thin film transistor illustrated in FIGS. 8A to 8D, FIGS. 9A to 9C and FIGS. 10A to 10D, an opening for connection between a semiconductor layer and a wiring is formed in a self-aligned manner.

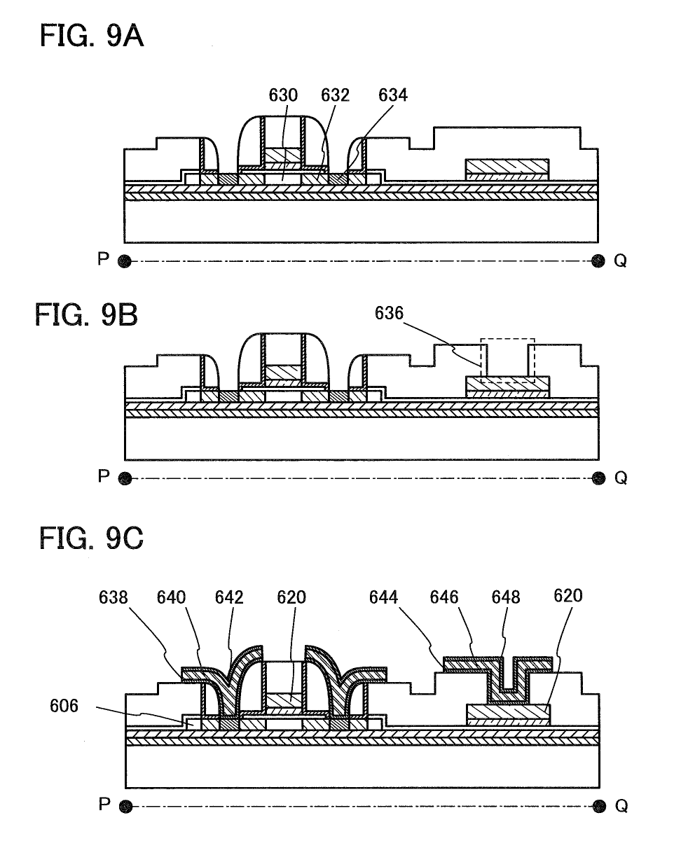

First, a semiconductor substrate manufactured by the semiconductor substrate manufacturing appratus in any of the above-described embodiment modes, or the like is prepared (not illustrated). Then, a single crystal semiconductor layer in the semiconductor substrate is patterned into an island shape to form an island-shaped semiconductor layer 606, and then an insulating layer 608 serving as a gate insulating layer and a conductive layer serving as a gate electrode (or a wiring) are formed in this order. In FIGS. 8A to 8D and FIGS. 9A to 9C, the conductive layer serving as a gate electrode is formed with a two layer structure; however, this is not a limiting example. Here, the insulating layer 608 can be formed using a material such as silicon oxide, silicon oxynitride, silicon nitride oxide or silicon nitride by a CVD method, a sputtering method, or the like. The thickness of the insulating layer 608 may be from about 5 nm to about 100 nm. The conductive layer can be formed using a material such as tantalum (Ta), tungsten (W), titanium (Ti), molybdenum (Mo), aluminum (Al), copper (Cu), chromium (Cr), or niobium (Nb) by a CVD method, a sputtering method, or the like. The total thickness of the two layers of the conductive layer may be from about 100 nm to about 500 nm. Note that in this embodiment mode, a case will be described in which the insulating layer 608 is formed using silicon oxide (with a thickness of 20 nm), the conductive layer (a lower layer) is formed using tantalum nitride (with a thickness of 50 nm) and the conductive layer (an upper layer) is formed using tungsten (with a thickness of 200 nm).

Note that in order to control the threshold voltage of the thin film transistor, an impurity imparting a p-type conductivity, such as boron, aluminum, or gallium or an impurity imparting an n-type conductivity, such as phosphorus or arsenic, may be added into the above-described semiconductor layer. For example, in the case of adding boron as an impurity imparting p-type conductivity, boron may be added at a concentration of from 5×10¹⁶ cm⁻³ to 1×10¹⁷ cm⁻³. Further, hydrogenation treatment may be performed on the semiconductor layer. The hydrogenation treatment is performed for example, at 350° C. for approximately two hours in a hydrogen atmosphere.

Next, the conductive layer serving as a gate electrode is patterned. Note that in a manufacturing method of a thin film transistor in FIGS. 8A to 8D, FIGS. 9A to 9C and FIGS. 10A to 10D, at least two patterning steps are performed on the conductive layer, and here, a first patterning step is performed. As a result of this, a conductive layer 610 and a conductive layer 612 which are slightly larger than the gate electrode which is to be formed finally are formed. Here, the phrase “slightly larger” herein means a size with which a resist mask for forming the gate electrode in a second patterning step can be formed in accordance with the position of the conductive layer 610 and the conductive layer 612. Note that the two patterning steps may be performed on a region overlapping with the island-shape semiconductor layer 606 which is a conductive layer and the two patterning steps do not need to be performed on an entire surface of the conductive layer.

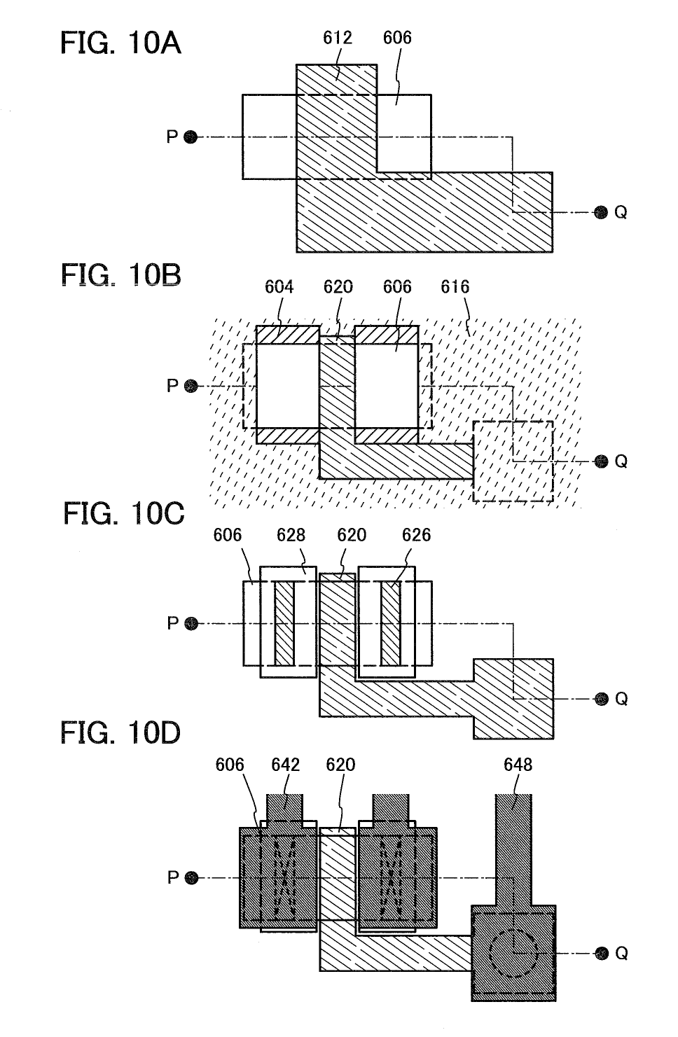

After that, an insulating layer 614 is formed so as to cover the insulating layer 608, the conductive layer 610, and the conductive layer 612 (see FIG. 8A and FIG. 10A). Here, the insulating layer 614 can be formed using a material such as silicon oxide, silicon oxynitride, silicon nitride oxide, silicon nitride, hafnium oxide or aluminum oxide by a CVD method, a sputtering method, or the like. The thickness of the insulating layer 614 is preferably from about 0.5 μm to about 2 μm. As an example, a case where the insulating layer 614 is formed using silicon oxide (with a thickness of 1 μm) will be described. Note that in FIGS. 8A to 8D, FIGS. 9A to 9C and FIGS. 10A to 10D a semiconductor substrate having a structure in which an insulating layer 602, an insulating layer 604, and the semiconductor layer are formed in this order over a base substrate 600 is described; however, this structure is not a limiting example.

Note that FIG. 8A is a view corresponding to a cross section taken along a line P-Q of FIG. 10A which is a plane view. Similarly, FIG. 8B, FIG. 8D and FIG. 9C are views corresponding to cross sections taken along lines P-Q of FIG. 10B, FIG. 10C and FIG. 10D, respectively. In the plane views illustrated in FIG. 10A to 10D, some components in the corresponding cross-sectional views are omitted for simplicity.

Next, a resist mask 616 for forming a gate electrode, which is used in patterning, is formed over the insulating layer 614. This patterning corresponds to the second patterning step of the above-described patterning steps which are performed on the conductive layer. The resist mask 616 can be formed in such a manner that a resist material which is a photosensitive substance is applied, and then a pattern is exposed to light. After formation of the resist mask 616, the conductive layer 610, the conductive layer 612 and the insulating layer 614 are patterned with use of the resist mask 616. Specifically, the insulating layer 614 is selectively etched to form an insulating layer 622, and then the conductive layer 610 and the conductive layer 612 are selectively etched to form a conductive layer 618 and a conductive layer 620 which serve as a gate electrode (see FIG. 8B and FIG. 10B). Here, when the insulating layer 614 is selectively etched, part of the insulating layer 608 which serves as a gate insulating layer is also etched at the same time.

Next, the resist mask 616 is removed, and then an insulating layer 624 is formed so as to cover the island-shaped semiconductor layer 606, the insulating layer 608, the conductive layer 618, the conductive layer 620, the insulating layer 622, and the like. The insulating layer 624 serves as a barrier layer at the time of forming sidewalls later. Although the insulating layer 624 can be formed using a material such as silicon oxide, silicon oxynitride, silicon nitride oxide, silicon nitride, hafnium oxide, aluminum oxide or tantalum oxide, it is preferable to form the insulating layer 624 using a material having etching selectivity with respect to a material used for the sidewalls later in order to make the insulating layer 624 serve as a barrier layer. The thickness of the insulating layer 624 may be from about 10 nm to about 200 nm. In this embodiment mode, the insulating layer 624 is formed using silicon nitride (with a thickness of 50 nm).

After formation of the insulating layer 624, an impurity element imparting one conductivity type is added to the island-shaped semiconductor layer 606 using the conductive layer 618, the conductive layer 620, the insulating layer 622, or the like as a mask. In this embodiment mode, an impurity element imparting n-type conductivity (e.g., phosphorus or arsenic) is added to the island-shaped semiconductor layer 606. By addition of the impurity element, impurity regions 626 are formed in the island-shaped semiconductor layer 606 (see FIG. 8C). Note that in this embodiment mode, after formation of the insulating layer 624, an impurity element imparting n-type conductivity is added; however, this is not a limiting example. For example, after or before removal of the resist mask, the impurity element may be added, and then the insulating layer 624 may be formed. An impurity element to be added can also be an impurity element imparting p-type conductivity.

Next, sidewalls 628 are formed (see FIG. 8D and FIG. 10C). The sidewalls 628 can be formed in such a manner that an insulating layer is formed so as to cover the insulating layer 624 and anisotropic etching mainly in a perpendicular direction is performed on the insulating layer. This is because the insulating layer is selectively etched by the anisotropic etching. The insulating layer can be formed using a material such as silicon oxide, silicon oxynitride, silicon nitride oxide, silicon nitride, hafnium oxide, aluminum oxide or tantalum oxide by a CVD method, a sputtering method, or the like. Further, a film containing an organic material may be formed by a spin coating method, or the like. In this embodiment mode, silicon oxide is used as a material for the insulating layer. That is, the sidewalls 628 are formed using silicon oxide. In addition, as an etching gas, a mixed gas of CHF₃ and helium can be used, for example. Note that the step of forming the sidewalls 628 is not limited to this example.

Next, an impurity element imparting one conductivity type is added to the island-shaped semiconductor layer 606 using the insulating layer 622, the sidewalls 628, or the like as a mask. Note that the impurity element imparting one conductivity type added to the island-shaped semiconductor layer 606 at this time has the same conductivity type as the impurity element that has been added to the island-shaped semiconductor layer 606 in the previous step, and is added at a higher concentration than that of the impurity element added in the previous step. That is, in this embodiment mode, an impurity element imparting n-type conductivity is added.

By addition of the impurity element, a channel formation region 630, low-concentration impurity regions 632, and high-concentration impurity regions 634 are formed in the island-shaped semiconductor layer 606. The low-concentration impurity regions 632 each serve as an LDD (lightly doped drain) region and the high-concentration impurity regions 634 each serve as a source or a drain.

Next, the insulating layer 624 is etched to form openings (contact holes) which reach the high-concentration impurity regions (see FIG. 9A). Since the insulating layer 622 and the sidewalls 628 are formed using silicon oxide and the insulating layer 624 is formed using silicon nitride in this embodiment mode, the insulating layer 624 can be selectively etched to form the openings.

After formation of the openings which reach the high-concentration impurity regions, the insulating layer 614 is selectively etched to form an opening 636 (see FIG. 9B). The opening 636 is formed larger than the opening which reaches the high-concentration impurity region. This is because a minimum line width of the opening 636 is determined in accordance with a process rule or a design rule, while the opening which reaches the high-concentration impurity region is miniaturized by being formed in a self-aligned manner.

After that, a conductive layer which is in contact with the high-concentration impurity regions 634 in the island-shaped semiconductor layer 606 and the conductive layer 620 is formed in the openings which reach the high-concentration impurity regions and the opening 636. The conductive layer can be formed by a CVD method, a sputtering method, or the like. As a material of the conductive layer, aluminum (Al), tungsten (W), titanium (Ti), tantalum (Ta), molybdenum (Mo), nickel (Ni), platinum (Pt), copper (Cu), gold (Au), silver (Ag), manganese (Mn), neodymium (Nd), carbon (C), silicon (Si), or the like can be used. Moreover, an alloy containing such metal as the main component or a compound containing such metal may be used. The conductive layer may have a single-layer structure or a stacked-layer structure. In this embodiment mode, a case is described in which the conductive layer has a three-layer structure of titanium, aluminum and titanium.

The conductive layer is selectively etched to form a conductive layer 638, a conductive layer 640 and a conductive layer 642 which serve as a source or drain electrode (a source or drain wiring), a conductive layer 644, a conductive layer 646 and a conductive layer 648 which are connected to the conductive layer 620 and serve as a wiring (see FIG. 9C and FIG. 10D). Through the process, a thin film transistor in which a connection between the island-shaped semiconductor layer 606 and the conductive layer serving as the source or drain electrode is formed in a self-aligned manner is completed.

Since connection of the source or drain electrode can be formed in a self-aligned manner by the method illustrated in FIGS. 8A to 8D, FIGS. 9A to 9C and FIGS. 10A to 10D, a structure of the transistor can be miniaturized. That is, the degree of integration of semiconductor elements can be increased. Further, since a length of the channel or the low-concentration impurity region can be determined in a self-aligned manner, variation in channel resistance, which is a problem in miniaturization, can be suppressed. That is, a transistor with excellent characteristics can be provided.

Although the method for manufacturing the TFT is described with reference to FIGS. 6A to 6D, FIGS. 7A to 7C, FIGS. 8A to 8D, FIGS. 9A to 9C, and FIGS. 10A to 10D, a semiconductor device with higher added value can be manufactured by formation of a variety of semiconductor elements such as a capacitor and a resistor together with the TFT. Hereinafter, a specific mode of the semiconductor device is described with reference to drawings.

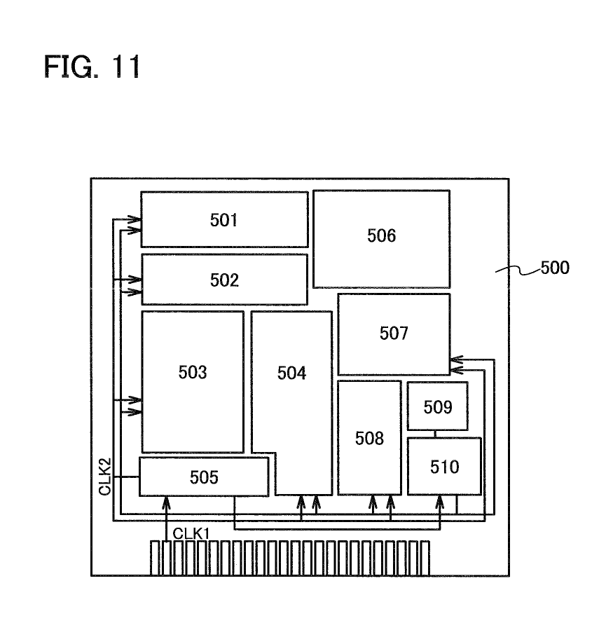

First, a microprocessor will be described as an example of a semiconductor device. FIG. 11 is a block diagram illustrating a structural example of a microprocessor 500.

The microprocessor 500 has an arithmetic logic unit (also referred to as an “ALU”) 501, an arithmetic logic unit controller (ALU controller) 502, an instruction decoder 503, an interrupt controller 504, a timing controller 505, a register 506, a register controller 507, a bus interface (Bus I/F) 508, a read only memory 509, and a memory interface 510.

An instruction inputted to the microprocessor 500 via the bus interface 508 is inputted to the instruction decoder 503 and decoded. Then, the instruction is inputted to the arithmetic logic unit controller 502, the interrupt controller 504, the register controller 507, and the timing controller 505. The arithmetic logic unit controller 502, the interrupt controller 504, the register controller 507, and the timing controller 505 perform various controls based on the decoded instruction.

The arithmetic logic unit controller 502 generates a signal for controlling the operation of the arithmetic logic unit 501. While the microprocessor 500 is executing a program, the interrupt controller 504 processes an interrupt request from an external input and output device or a peripheral circuit. The interrupt controller 504 judges the priority of the interrupt request or a mask state, and processes the interrupt request. The register controller 507 generates an address of the register 506, and reads and writes data from and to the register 506 in accordance with the state of the microprocessor 500. The timing controller 505 generates signals for controlling timing of driving of the arithmetic logic unit 501, the arithmetic logic unit controller 502, the instruction decoder 503, the interrupt controller 504, and the register controller 507. For example, the timing controller 505 is provided with an internal clock generator for generating an internal clock signal CLK2 based on a reference clock signal CLK1. As illustrated in FIG. 11, the internal clock signal CLK2 is inputted to another circuit.

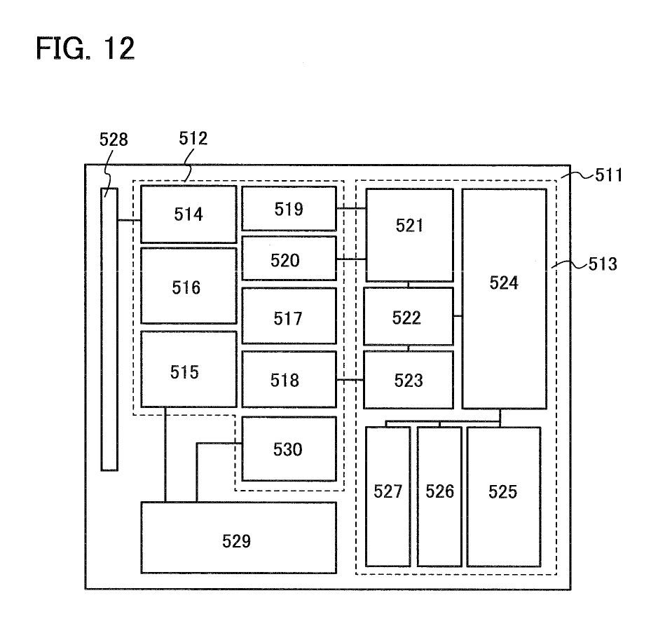

Next, an example of a semiconductor device having a function of transmitting and receiving data wirelessly and also having an arithmetic function is described. FIG. 12 is a block diagram illustrating a structural example of such a semiconductor device. The semiconductor device illustrated in FIG. 12 can be referred to as a computer which operates by transmitting and receiving signals to and from an external device by wireless communication (hereinafter the device is referred to as an “RFCPU”).

As illustrated in FIG. 12, an RFCPU 511 has an analog circuit portion 512 and a digital circuit portion 513. The analog circuit portion 512 has a resonance circuit 514 having a resonant capacitor, a rectifier circuit 515, a constant voltage circuit 516, a reset circuit 517, an oscillator circuit 518, a demodulation circuit 519, and a modulation circuit 520. The digital circuit portion 513 has an RF interface 521, a control register 522, a clock controller 523, a CPU interface 524, a central processing unit 525, a random access memory 526, and a read only memory 527.

The operation of the RFCPU 511 is roughly described below. The RFCPU 511 receives power from an antenna. The power is amplified by the resonance circuit 514 and is stored in a capacitor portion 529 via the rectifier circuit 515. The capacitor portion 529 is preferably formed using a capacitor such as a ceramic capacitor or an electric double layer capacitor. The capacitor portion 529 is not necessarily integrated over the same substrate as the RFCPU 511 and may be incorporated into the RFCPU 511 as another component.

The reset circuit 517 generates a signal which resets the digital circuit portion 513 to be initialized. For example, a signal which rises after an increase in a power supply voltage is generated as a reset signal. The oscillator circuit 518 changes the frequency and the duty ratio of a clock signal in accordance with a control signal generated by the constant voltage circuit 516. The demodulation circuit 519 demodulates a received signal, and the modulation circuit 520 modulates data to be transmitted.

For example, the demodulation circuit 519 is formed using a low-pass filter and binarizes a received signal of an amplitude shift keying (ASK) system based on variation of the amplitude. The modulation circuit 520 transmits transmission data by changing the amplitude of a transmission signal of the amplitude shift keying (ASK) system. The modulation circuit 520 changes the resonance point of the resonance circuit 514, whereby the amplitude of a communication signal is changed.

The clock controller 523 generates a control signal for changing the frequency and the duty ratio of the clock signal in accordance with the power supply voltage or current consumption in the central processing unit 525. The power supply voltage is monitored by the power supply control circuit 530.

A signal which is inputted to the RFCPU 511 from the antenna 528 is demodulated by the demodulation circuit 519, and then divided into a control command, data, and the like by the RF interface 521. The control command is stored in the control register 522. The control command includes reading of data stored in the read only memory 527, writing of data to the random access memory 526, an arithmetic instruction to the central processing unit 525, and the like.

The central processing unit 525 accesses the read only memory 527, the random access memory 526, and the control register 522 via the CPU interface 524. The CPU interface 524 has a function of generating an access signal for any one of the read only memory 527, the random access memory 526, and the control register 522 based on an address requested by the central processing unit 525.

As an arithmetic method of the central processing unit 525, a method can be employed in which the read only memory 527 stores an operating system (OS) and a program is read at the time of starting operation and then is executed. Alternatively, a method can be employed in which a circuit dedicated to arithmetic is formed as an arithmetic circuit and an arithmetic processing is conducted using hardware. In a method in which both hardware and software are used, part of arithmetic processing can be conducted by a circuit dedicated to arithmetic, and the other part of the arithmetic processing can be conducted by the central processing unit 525 with use of a program.

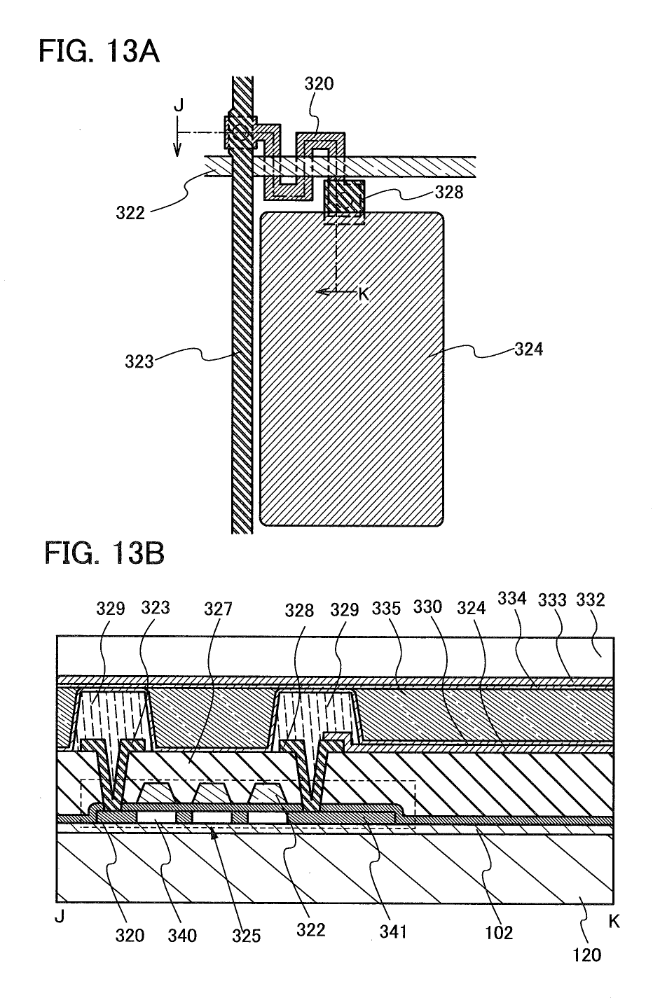

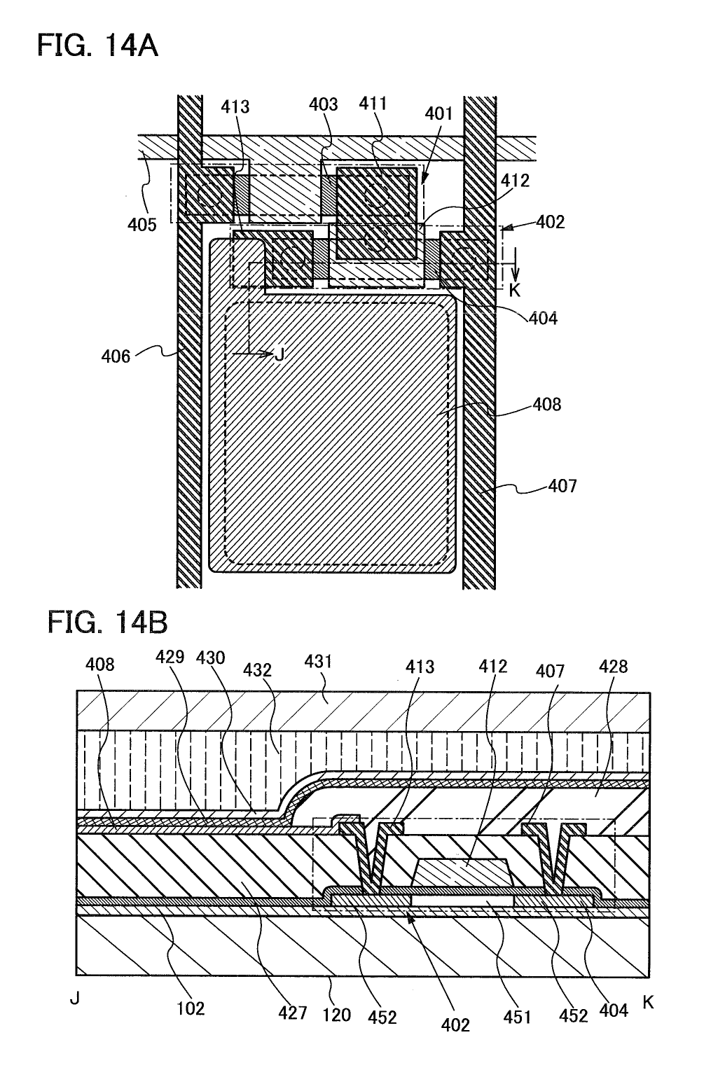

Next, a display device will be described as a semiconductor device with reference to FIGS. 13A and 13B, and FIGS. 14A and 14B.

FIGS. 13A and 13B are drawings for describing a liquid crystal display device. FIG. 13A is a plan view of a pixel of a liquid crystal display device. FIG. 13B is a cross-sectional view taken along a line J-K of FIG. 13A.

As illustrated in FIG. 13A, a pixel includes a single crystal semiconductor layer 320, a scan line 322 intersecting with the single crystal semiconductor layer 320, a signal line 323 intersecting with the scan line 322, a pixel electrode 324, and an electrode 328 which electrically connects the pixel electrode 324 to the single crystal semiconductor layer 320. The single crystal semiconductor layer 320 is formed using the single crystal semiconductor layer 124 bonded to the semiconductor substrate by using a semiconductor substrate manufacturing appratus described in any of the above-described embodiment modes and is included in a TFT 325 of the pixel.

As illustrated in FIG. 13B, the insulating film 102 and the single crystal semiconductor layer 320 are stacked over the base substrate 120. The base substrate 120 is made of glass. The single crystal semiconductor layer 124 of the semiconductor substrate is separated into each element by etching, whereby the single crystal semiconductor layer 320 of the TFT 325 is formed. A channel formation region 340 and n-type high-concentration impurity regions 341 into which donors are added are formed in the single crystal semiconductor layer 320. A gate electrode of the TFT 325 is included in the scan line 322 and one of a source electrode and a drain electrode of the TFT 325 is included in the signal line 323.

The signal line 323, the pixel electrode 324, and the electrode 328 are provided over an interlayer insulating film 327. Column spacers 329 are formed over the interlayer insulating film 327. An alignment film 330 is formed to cover the signal line 323, the pixel electrode 324, the electrode 328, and the column spacers 329. A counter substrate 332 is provided with a counter electrode 333 and an alignment film 334 which covers the counter electrode 333. The column spacers 329 are formed to keep the space between the base substrate 120 and the counter substrate 332. A liquid crystal layer 335 is formed in the space which is formed by the column spacers 329. A step is generated on the interlayer insulating film 327 at the connection portion between the high-concentration impurity regions 341, and the signal line 323 and the electrode 328 due to formation of contact holes; therefore, alignment of liquid crystals in the liquid crystal layer 335 tends to be disordered at this connection portion. Therefore, the column spacers 329 are formed at the step portions to prevent the disorder of the alignment of liquid crystals.

Next, an electroluminescent display device (hereinafter referred to as an “EL display device”) will be described with reference to FIGS. 14A and 14B. FIG. 14A is a plan view of a pixel of an EL display device. FIG. 14B is a cross-sectional view taken along a line J-K of FIG. 14A.

As illustrated in FIG. 14A, a pixel includes a scan line 405, a signal line 406, a current supply line 407, a pixel electrode 408, and a selection transistor 401 and a display control transistor 402 each including a TFT. Each pixel is provided with a light-emitting element having a structure in which a layer containing an electroluminescent material (an EL layer) is sandwiched between a pair of electrodes. One electrode of the light-emitting element is the pixel electrode 408. Further, in a semiconductor film 403, a channel formation region, a source region, and a drain region of the selection transistor 401 are formed. Further, in a semiconductor film 404, a channel formation region, a source region, and a drain region of the display control transistor 402 are formed. The semiconductor films 403 and 404 are formed using the single crystal semiconductor layer 124 bonded to the semiconductor substrate manufactured by using a semiconductor substrate manufacturing appratus described in any of the above-described embodiment modes.

In the selection transistor 401, a gate electrode is included in the scan line 405, one of a source electrode and a drain electrode is included in the signal line 406, and the other thereof is formed as an electrode 411. In the display control transistor 402, a gate electrode 412 is electrically connected to the electrode 411, one of a source electrode and a drain electrode is formed as an electrode 413 which is electrically connected to the pixel electrode 408, and the other thereof is included in the current supply line 407.

The display control transistor 402 is a p-channel TFT. As illustrated in FIG. 14B, a channel formation region 451 and p-type high-concentration impurity regions 452 are formed in the semiconductor film 404.

An interlayer insulating film 427 is formed so as to cover the gate electrode 412 of the display control transistor 402. Over the interlayer insulating film 427, the signal line 406, the current supply line 407, the electrode 411, the electrode 413, and the like are formed. Over the interlayer insulating film 427, the pixel electrode 408 which is electrically connected to the electrode 413 is formed. A peripheral portion of the pixel electrode 408 is surrounded by a partition wall layer 428 which has an insulating property. An EL layer 429 is formed over the pixel electrode 408, and a counter electrode 430 is formed over the EL layer 429. A counter substrate 431 is provided as a reinforcing plate and is fixed to the base substrate 120 by a resin layer 432.

The grayscale of the EL display device is controlled by either a current driving method by which the luminance of the light-emitting element is controlled by current or a voltage driving method by which the luminance of the light-emitting element is controlled by voltage. The current driving method is difficult to be employed when characteristics of a transistor in each pixel are largely different from transistors in other pixels, and thus a compensation circuit for compensating the variation in the characteristics is needed. The EL display device is manufactured by a manufacturing process of a semiconductor substrate according to any of the above embodiment modes and a manufacturing method including a gettering step, so that the selection transistor 401 and the display control transistor 402 do not have variation in characteristics in each pixel. Thus, the current driving method can be employed.

That is, a wide variety of electronic devices can be manufactured using a semiconductor device manufactured according to any of the above-described embodiment modes. The electronic devices include, in their category, cameras such as video cameras, digital cameras, navigation systems, audio reproducing devices (such as car audios or audio components), computers, game machines, portable information terminals (such as mobile computers, mobile phones, portable game machines, or e-book readers), and image reproducing devices having storage media (specifically, devices provided with display devices capable of playing audio data stored in recording media such as digital versatile disk (DVD) and displaying stored image data).

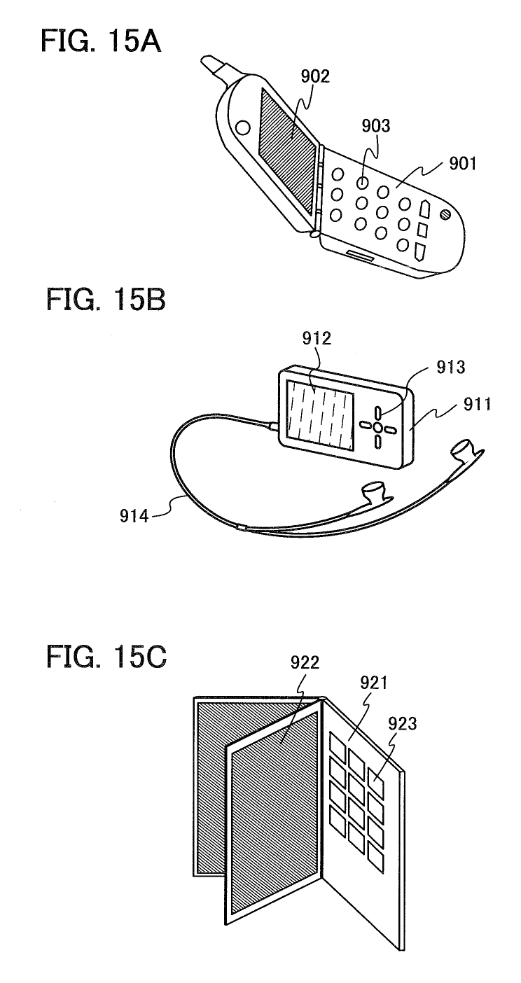

Specific modes of the electronic devices are described with reference to FIGS. 15A to 15C. FIG. 15A is an external view illustrating an example of a mobile phone 901. This mobile phone 901 includes a display portion 902, operation switches 903, and the like. The liquid crystal display device illustrated in FIGS. 13A and 13B or the EL display device illustrated in FIGS. 14A and 14B is applied to the display portion 902, whereby the display portion 902 can have little display unevenness and excellent image quality.

FIG. 15B is an external view illustrating a structural example of a digital player 911. The digital player 911 includes a display portion 912, an operation portion 913, a pair of earphones 914, and the like. The pair of earphones 914 can be replaced by headphones or wireless earphones. The liquid crystal display device illustrated in FIGS. 13A and 13B or the EL display device illustrated in FIGS. 14A and 14B is applied to the display portion 912, whereby the display portion 912 can display high-definition images and a large amount of textual information even when the screen size ranges approximately from 0.3 inches to 2 inches.

FIG. 15C is an external view of an e-book reader 921. This e-book reader 921 includes a display portion 922 and operation switches 923. The e-book reader 921 may incorporate a modem or may incorporate the RFCPU illustrated in FIG. 12 so that information can be transmitted and received wirelessly. The liquid crystal display device illustrated in FIGS. 13A and 13B or the EL display device illustrated in FIGS. 14A and 14B is applied to the display portion 922, whereby the display portion 922 can display high-quality images.

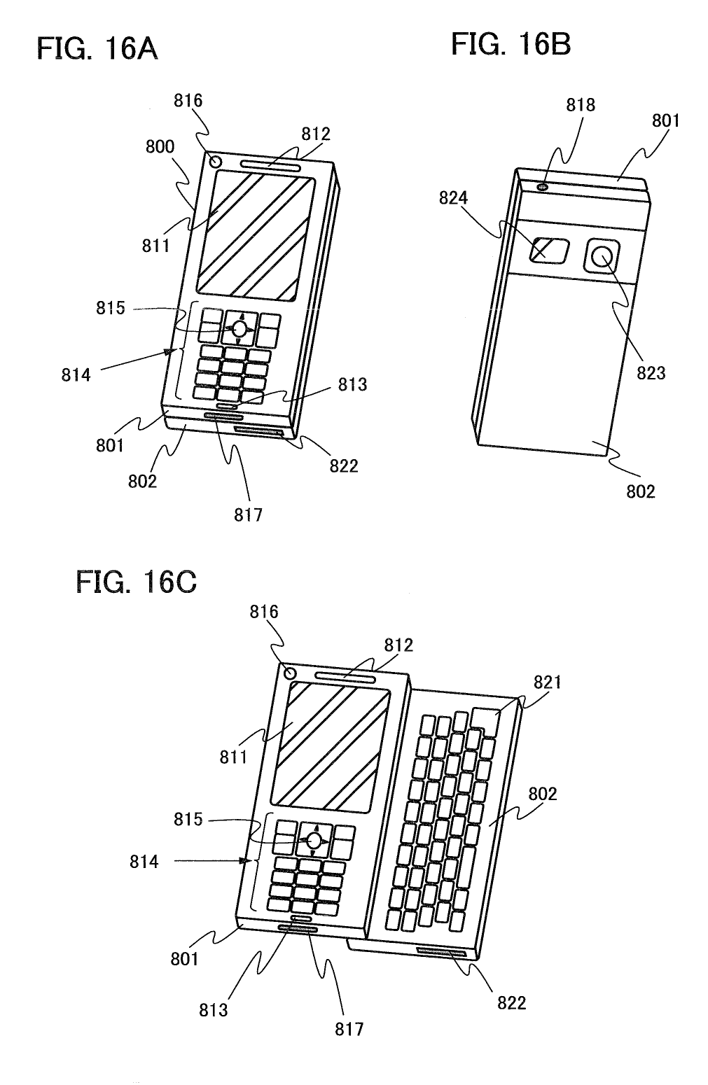

FIGS. 16A to 16C exemplifies a structure of a mobile phone 800 to which a semiconductor device of any of the above embodiment modes is applied. FIG. 16A is a front view, FIG. 16B is a rear view, and FIG. 16C is a development view. The mobile phone 800 is a so-called smartphone which has both functions of a mobile phone and a portable information terminal, is incorporated with a computer, and can perform a variety of data processing in addition to voice calls.

The mobile phone 800 includes two housings 801 and 802. The housing 801 includes a display portion 811, a speaker 812, a microphone 813, operation keys 814, a pointing device 815, a camera lens 816, an external connection terminal 817, and the like, while the housing 802 includes a keyboard 821, an external memory slot 822, a camera lens 823, a light 824, an earphone terminal 818, and the like. In addition, an antenna is incorporated in the housing 801. When the liquid crystal display device illustrated in FIGS. 13A and 13B or the EL display device illustrated in FIGS. 14A and 14B is applied to the display portion 811, the display portion 811 can have little display unevenness and excellent image quality.

Further, in addition to the above structure, the mobile phone 800 may incorporate a non-contact IC chip, a small size memory device, or the like.

The display direction of the display portion 811 can be changed depending on a usage pattern. The mobile phone 800 is provided with the camera lens 816 on the same surface as the display portion 811, and thus it can be used as a video phone. Further, a still image and a moving image can be taken with the camera lens 823 and the light 824 by using the display portion 811 as a viewfinder. The speaker 812 and the microphone 813 are not limited to use for verbal communication, and can be used for a videophone, recording, reproduction, and the like. With use of the operation keys 814, operation of incoming and outgoing calls, easy input of information such as electronic mails or the like, scrolling of a screen, cursor motion and the like are possible. Furthermore, the housing 801 and the housing 802 (FIG. 16A), which are overlapped with each other, are slid and developed as illustrated in FIG. 16C, and the mobile phone 800 can be used as a portable information terminal. In this case, smooth operation is possible with use of the keyboard 821 and the pointing device 815. The external connection terminal 817 can be connected to an AC adapter and various types of cables such as a USB cable, and charging and data communication with a personal computer are possible. Moreover, a large amount of data can be stored and transferred by using a storage medium inserted into the external memory slot 822.

Further, in addition to the above-described functions, the mobile phone 800 may also have an infrared communication function, a television reception function, or the like.

In the above-described manner, electronic devices or lighting can be obtained by application of a semiconductor substrate manufactured according to any of the above embodiment modes. As described above, a semiconductor substrate manufactured according to any of the above embodiment modes has a remarkably wide application range, and can be applied to electronic devices in a wide variety of fields.

This application is based on Japanese Patent Application serial no. 2008-007077 filed with Japan Patent Office on Jan. 16, 2008, the entire contents of which are hereby incorporated by reference.

1. An apparatus for manufacturing a semiconductor substrate in which a single crystal semiconductor layer separated from a single crystal semiconductor substrate is provided over a base substrate having an insulating surface, comprising:
a cleaning portion in which a bonding surface of the base substrate, and a bonding surface of the single crystal semiconductor substrate are cleaned, wherein the single crystal semiconductor substrate has a surface provided with an insulating layer serving as a bonding layer and includes an embrittlement region provided in a region at a predetermined depth from the surface;
an electromagnetic wave irradiation portion in which the base substrate and the single crystal semiconductor substrate are attached to each other, the single crystal semiconductor substrate is irradiated with an electromagnetic wave having a frequency of 300 MHz to 300 GHz to be heated, and the single crystal semiconductor substrate is separated using the embrittlement region as a separation plane, so that the single crystal semiconductor layer separated from the single crystal semiconductor substrate is fixed to the base substrate; and
a heat treatment portion in which the single crystal semiconductor layer fixed to the base substrate is subjected to heat treatment,
wherein the electromagnetic wave irradiation portion is connected to the cleaning portion, and
the heat treatment portion is connected to the electromagnetic wave irradiation portion.

2. The apparatus according to claim 1, further comprising a reprocessing portion of the single crystal semiconductor substrate, in which a surface of the single crystal semiconductor substrate from which the single crystal semiconductor layer is separated, is planarized.

3. The apparatus according to claim 1, wherein in the heat treatment portion, the single crystal semiconductor layer is heated at 500° C. or higher.

4. The apparatus according to claim 1, wherein the electromagnetic wave irradiation portion comprises:
a plurality of waveguides arranged in parallel;
a plurality of slots provided for each of the plurality of waveguides; and
a dielectric provided to correspond to and be in contact with each of the plurality of slots.

5. The apparatus according to claim 4, wherein the plurality of slots are provided at an interval between adjacent slots, and the interval is half of a wavelength of the electromagnetic wave transmitted in the waveguides.

6. The apparatus according to claim 1, wherein the electromagnetic wave irradiation portion comprises:
a plurality of waveguides;
a plurality of slots provided for each of the plurality of waveguides;
a dielectric provided to correspond to and be in contact with each of the plurality of slots; and
a stage which can move in a plane direction.

7. The apparatus according to claim 6, wherein the plurality of slots are provided at an interval between adjacent slots, and the interval is half of a wavelength of the electromagnetic wave transmitted in the waveguides.

8. The apparatus according to claim 1, wherein the electromagnetic wave irradiation portion comprises:
a plurality of waveguides;
a plurality of slots provided for each of the plurality of waveguides;
a dielectric provided to correspond to and be in contact with each of the plurality of slots;
a stage having a plurality of gas blowing holes;
a gas blower for supplying a gas to each of the gas blowing holes; and
transfer rollers arranged on both sides of the stage.

9. The apparatus according to claim 8, wherein the plurality of slots are provided at an interval between adjacent slots, and the interval is half of a wavelength of the electromagnetic wave transmitted in the waveguides.

10. The apparatus according to claim 1 further comprising:
a first arm for holding the single crystal semiconductor substrate, and
a second arm for holding the base substrate.

11. The apparatus according to claim 1,
wherein the electromagnetic wave irradiation portion is directly connected to the cleaning portion, and
the heat treatment portion is directly connected to the electromagnetic wave irradiation portion.

12. An apparatus for manufacturing a semiconductor substrate in which a plurality of single crystal semiconductor layers separated from a plurality of single crystal semiconductor substrates are provided over a base substrate having an insulating surface, comprising:
a cleaning portion in which a bonding surface of the base substrate, and a bonding surface of each of the plurality of single crystal semiconductor substrates are cleaned, wherein each of the plurality of single crystal semiconductor substrates has a surface provided with an insulating layer serving as a bonding layer and includes an embrittlement region provided in a region at a predetermined depth from the surface;
an electromagnetic wave irradiation portion in which the base substrate and the plurality of single crystal semiconductor substrates are attached to each other, the plurality of single crystal semiconductor substrates are each irradiated with an electromagnetic wave having a frequency of 300 MHz to 300 GHz to be heated, and the plurality of single crystal semiconductor substrates are each separated using the embrittlement region as a separation plane, so that the plurality of single crystal semiconductor layers separated from the plurality of single crystal semiconductor substrates are fixed to the base substrate; and
a heat treatment portion in which the plurality of single crystal semiconductor layers fixed to the base substrate are subjected to heat treatment,
wherein the electromagnetic wave irradiation portion is connected to the cleaning portion, and
the heat treatment portion is connected to the electromagnetic wave irradiation portion.

13. The apparatus according to claim 12, further comprising a reprocessing portion of the plurality of single crystal semiconductor substrates, in which a surface of each of the plurality of single crystal semiconductor substrates from which the plurality of single crystal semiconductor layers are separated, is planarized.

14. The apparatus according to claim 12, wherein in the heat treatment portion, the plurality of single crystal semiconductor layers are heated at 500° C. or higher.

15. The apparatus according to claim 12, wherein the electromagnetic wave irradiation portion comprises:
a plurality of waveguides arranged in parallel;
a plurality of slots provided for each of the plurality of waveguides; and
a dielectric provided to correspond to and be in contact with each of the plurality of slots.

16. The apparatus according to claim 15, wherein the plurality of slots are provided at an interval between adjacent slots, and the interval is half of a wavelength of the electromagnetic wave transmitted in the waveguides.

17. The apparatus according to claim 12, wherein the electromagnetic wave irradiation portion comprises:
a plurality of waveguides;
a plurality of slots provided for each of the plurality of waveguides;
a dielectric provided to correspond to and be in contact with each of the plurality of slots; and
a stage which can move in a plane direction.

18. The apparatus according to claim 17, wherein the plurality of slots are provided at an interval between adjacent slots, and the interval is half of a wavelength of the electromagnetic wave transmitted in the waveguides.

19. The apparatus according to claim 12, wherein the electromagnetic wave irradiation portion comprises:
a plurality of waveguides;
a plurality of slots provided for each of the plurality of waveguides;
a dielectric provided to correspond to and be in contact with each of the plurality of slots;
a stage having a plurality of gas blowing holes;
a gas blower for supplying a gas to each of the gas blowing holes; and
transfer rollers arranged on both sides of the stage.

20. The apparatus according to claim 19, wherein the plurality of slots are provided at an interval between adjacent slots, and the interval is half of a wavelength of the electromagnetic wave transmitted in the waveguides.

21. The apparatus according to claim 12 further comprising:
a first arm for holding the single crystal semiconductor substrate, and
a second arm for holding the base substrate.

22. The apparatus according to claim 12,
wherein the electromagnetic wave irradiation portion is directly connected to the cleaning portion, and
the heat treatment portion is directly connected to the electromagnetic wave irradiation portion.

23. (canceled)

24. (canceled)
